(19) United States
(12) Patent Application Publication
(10) Pub. No.: US 2003/0236439 A1
(43) Pub. Date: Dec. 25, 2003

(54) Non-steroidal analogs of 2-methoxyestradiol

(76) Inventors: Gregory Agoston, Germantown, MD (US); Jamshed H. Shah, Brookeville, MD (US); Kimberly A. Hunsucker, Alpharetta, CA (US); Anthony M. Treston, Rockville, MD (US); Victor S. Pribluda, Silver Spring, MD (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/354,927

(22) Filed: Jan. 30, 2003

Related U.S. Application Data
(60) Provisional application No. 60/354,046, filed on Jan. 30, 2002.

(51) Int. Cl. C07C 41/00; C07C 43/02; C07C 43/20

(52) U.S. Cl. 568/632; 568/633

(57) ABSTRACT

Compounds, compositions, and methods for treating disease states characterized by undesirable angiogenesis, proliferative activity, or cell mitosis by administering non-steroidal analogs of 2-methoxyestradiol of the general formula:

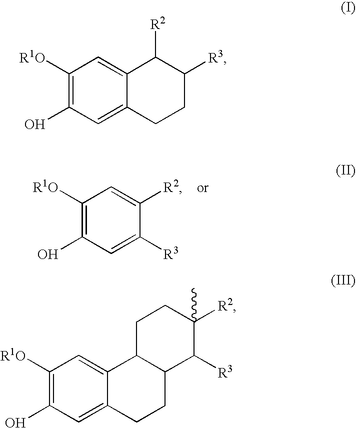

wherein R¹, R², and R³ are defined in the specification, are disclosed herein.

### PRIOR RELATED U.S. APPLICATION DATA

This application claims priority to U.S. Provisional Patent Application Serial No. 60/354,046 filed Jan. 30, 2002, the entirety of which is incorporated herein by reference.

### TECHNICAL FIELD OF THE INVENTION

This invention relates to compounds, compositions, and methods for treating disease states characterized by abnormal angiogenesis, abnormal proliferative activity, abnormal cell mitosis, or a combination of these events. More particularly, the present invention relates to non-steroidal analogs of 2-methoxyestradiol (2ME₂) and their effect on diseases characterized by abnormal angiogenesis and/or abnormal proliferative activity, including their effect on tumors.

### BACKGROUND OF THE INVENTION

Angiogenesis is the generation of new blood vessels into a tissue or organ. Under normal physiological conditions, humans and animals undergo angiogenesis only in very specific, restricted situations. For example, angiogenesis is normally observed in wound healing, fetal and embryonal development, and formation of the corpus luteum, endometrium and placenta.

Angiogenesis is controlled through a highly regulated system of angiogenic stimulators and inhibitors. The control of angiogenesis has been found to be altered in certain disease states and, in many cases, pathological damage associated with the diseases is related to uncontrolled angiogenesis. Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. Endothelial cells, lining the lumen of blood vessels, then protrude through the basement membrane. Angiogenic stimulants induce the endothelial cells to migrate through the eroded basement membrane. The migrating cells form a “sprout” off the parent blood vessel where the endothelial cells undergo mitosis and proliferate. The endothelial sprouts merge with each other to form capillary loops, creating a new blood vessel.

Persistent, unregulated angiogenesis occurs in many disease states, tumor metastases, and abnormal growth or proliferation by endothelial cells. The diverse pathological disease states in which unregulated angiogenesis is present have been grouped together as angiogenic-dependent or angiogenic-associated diseases.

One example of a disease mediated by angiogenesis and proliferative activity is ocular neovascular disease. This disease is characterized by invasion of new blood vessels into the structures of the eye, such as the retina or cornea. It is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma, and retrolental fibroplasia. Other diseases associated with corneal neovascularization include, but are not limited to, epidemic keratoconjunctivitis, Vitamin A deficiency, contact lens overwear, atopic keratitis, superior limbic keratitis, and pterygium keratitis sicca. Other diseases associated with undesirable angiogenesis include Sjögren's syndrome, acne rosacea, phylectenulosis, syphilis, Mycobacteria infections, lipid degeneration, chemical burns, bacterial ulcers, fungal ulcers, Herpes simplex infection, Herpes zoster infections, protozoan infections, Kaposi's sarcoma, Mooren's ulcer, Terrien's marginal degeneration, marginal keratolysis, rheumatoid arthritis, systemic lupus, polyarteritis, trauma, Wegener's syndrome, sarcoidosis, scleritis, Stevens-Johnson's disease, pemphigoid, and radial keratotomy.

Diseases associated with retinal/choroidal neovascularization and endothelial proliferative activity include, but are not limited to, diabetic retinopathy, macular degeneration, sickle cell anemia, sarcoidosis, syphilis, pseudoxanthoma elasticum, Paget's disease, vein occlusion, artery occlusion, carotid obstructive disease, chronic uveitis/vitritis, Mycobacteria infections, lyme's disease, systemic lupus erythematosis, retinopathy of prematurity, Eales' disease, Behcet's disease, infections causing retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other eye-related diseases include, but are not limited to, diseases associated with rubeosis (neovascularization of the angle) and diseases caused by the abnormal proliferation of fibrovascular or fibrous tissue, including all forms of prolific vitreoretinopathy.

Another angiogenesis and proliferative activity-associated disease is rheumatoid arthritis. The blood vessels in the synovial lining of the joints undergo angiogenesis. In addition to forming new vascular networks, the endothelial cells release factors and reactive oxygen species that lead to pannus growth and cartilage destruction. Angiogenesis may also play a role in osteoarthritis. The activation of the chondrocytes by angiogenic-related factors contributes to the destruction of the joint. At a later stage, the angiogenic factors promote new bone growth. Therapeutic intervention that prevents the bone destruction could halt the progress of the disease and provide relief for persons suffering with arthritis.

Chronic inflammation may also involve pathological angiogenesis and proliferative activity. Such diseases as ulcerative colitis and Crohn's disease show histological changes with the ingrowth of new blood vessels and the inflamed tissues. Bartonelosis, a bacterial infection found in South America, can result in a chronic stage that is characterized by proliferation of vascular endothelial cells. Another pathological role associated with angiogenesis is found in atherosclerosis. The plaques formed within the lumen of blood vessels have been shown to have angiogenic stimulatory activity.

The hypothesis that tumor growth is angiogenesis-dependent was first proposed in 1971. (Folkman, New Eng. J. Med., 285:1182-86 (1971)). In its simplest terms, this hypothesis states: “Once tumor ‘take’ has occurred, every increase in tumor cell population must be preceded by an increase in new capillaries converging on the tumor.” Tumor ‘take’ is currently understood to indicate a prevascular phase of tumor growth in which a population of tumor cells occupying a few cubic millimeters volume, and not exceeding a few million cells, can survive on existing host microvessels. Expansion of tumor volume beyond this phase requires the induction of new capillary blood vessels. For example, pulmonary micrometastases in the early prevascular phase in mice would be undetectable except by high power microscopy on histological sections.

Examples of the indirect evidence which support this concept include, but is not limited to, the following.

(1) The growth rate of tumors implanted in subcutaneous transparent chambers in mice is slow and linear before neovascularization, and rapid and nearly exponential after neovascularization. (Algire, et al., J. Nat. Cancer Inst., 6:73-85 (1945)).

(2) Tumors grown in isolated perfused organs where blood vessels do not proliferate are limited to 1-2 mm³ but expand rapidly to >1000 times this volume when they are transplanted to mice and become neovascularized. (Folkman, et al., Annals of Surgery, 164:491-502 (1966)).

(3) Tumor growth in the avascular cornea proceeds slowly and at a linear rate, but switches to exponential growth after neovascularization. (Gimbrone, Jr., et al., J. Nat. Cancer Inst., 52:421-27 (1974)).

(4) Tumors suspended in the aqueous fluid of the anterior chamber of the rabbit eye remain viable, avascular, and limited in size to <1 mm³. Once they are implanted on the iris vascular bed, they become neovascularized and grow rapidly, reaching 16,000 times their original volume within 2 weeks. (Gimbrone, Jr., et al., J. Exp. Med., 136:261-76).

(5) When tumors are implanted on the chick embryo chorioallantoic membrane, they grow slowly during an avascular phase of >72 hours, but do not exceed a mean diameter of 0.93+0.29 mm. Rapid tumor expansion occurs within 24 hours after the onset of neovascularization, and by day 7 these vascularized tumors reach a mean diameter of 8.0+2.5 mm. (Knighton, British J. Cancer, 35:347-56 (1977)).

(6) Vascular casts of metastases in the rabbit liver reveal heterogeneity in size of the metastases, but show a relatively uniform cut-off point for the size at which vascularization is present. Tumors are generally avascular up to 1 mm in diameter, but are neovascularized beyond that diameter. (Lien, et al., Surgery, 68:334-40 (1970)).

(7) In transgenic mice which develop carcinomas in the beta cells of the pancreatic islets, pre-vascular hyperplastic islets are limited in size to <1 mm. At 6-7 weeks of age, 4-10% of the islets become neovascularized, and from these islets arise large vascularized tumors of more than 1000 times the volume of the pre-vascular islets. (Folkman, et al., Nature, 339:58-61 (1989)).

(8) A specific antibody against VEGF (vascular endothelial growth factor) reduces microvessel density and causes “significant or dramatic” inhibition of growth of three human tumors which rely on VEGF as their sole mediator of angiogenesis (in nude mice). The antibody does not inhibit growth of the tumor cells in vitro. (Kim, et al., Nature, 362:841-44 (1993)).

(9) Anti-bFGF monoclonal antibody causes 70% inhibition of growth of a mouse tumor which is dependent upon secretion of bFGF as its only mediator of angiogenesis. The antibody does not inhibit growth of the tumor cells in vitro. (Hori, et al., Cancer Res., 51:6180-84 (1991)).

(10) Intraperitoneal injection of bFGF enhances growth of a primary tumor and its metastases by stimulating growth of capillary endothelial cells in the tumor. The tumor cells themselves lack receptors for bFGF, and bFGF is not a mitogen for the tumors cells in vitro. (Gross, et al., Proc. Am. Assoc. Cancer Res., 31:79 (1990)).

(11) A specific angiogenesis inhibitor (AGM-1470) inhibits tumor growth and metastases in vivo, but is much less active in inhibiting tumor cell proliferation in vitro. It inhibits vascular endothelial cell proliferation half-maximally at 4 logs lower concentration than it inhibits tumor cell proliferation. (Ingber, et al., Nature, 48:555-57 (1990)). There is also indirect clinical evidence that tumor growth is angiogenesis dependent.

(12) Human retinoblastomas that are metastatic to the vitreous develop into avascular spheroids which are restricted to less than 1 mm³ despite the fact that they are viable and incorporate ³H-thymidine (when removed from an enucleated eye and analyzed in vitro).

(13) Carcinoma of the ovary metastasizes to the peritoneal membrane as tiny avascular white seeds (1-3 mm³). These implants rarely grow larger until one or more of them becomes neovascularized.

(14) Intensity of neovascularization in breast cancer (Weidner, et al., New Eng. J. Med., 324:1-8 (1991); Weidner, et al., J Nat. Cancer Inst., 84:1875-87 (1992)) and in prostate cancer (Weidner, et al., Am. J. Pathol., 143(2):401-09 (1993)) correlates highly with risk of future metastasis.

(15) Metastasis from human cutaneous melanoma is rare prior to neovascularization. The onset of neovascularization leads to increased thickness of the lesion and an increased risk of metastasis. (Srivastava, et al., Am. J. Pathol., 133:419-23 (1988)).

(16) In bladder cancer, the urinary level of an angiogenic protein, bFGF, is a more sensitive indicator of status and extent of disease than is cytology. (Nguyen, et al., J. Nat. Cancer Inst., 85:241-42 (1993)).

Thus, it is clear that angiogenesis and endothelial cell proliferation play a major role in the metastasis of cancer. If this angiogenic activity could be repressed or eliminated, then the tumor, although present, would not grow. In the disease state, prevention of angiogenesis could avert the damage caused by the invasion of the new microvascular system. Therapies directed at control of the angiogenic processes could lead to the abrogation or mitigation of these diseases.

Angiogenesis and endothelium proliferation have been associated with a number of different types of cancer, including solid tumors and blood-borne tumors. Solid tumors with which angiogenesis has been associated include, but are not limited to, rhabdomyosarcomas, retinoblastoma, Ewing's sarcoma, neuroblastoma, and osteosarcoma. Angiogenesis is also associated with blood-borne tumors, such as leukemias, any of various acute or chronic neoplastic diseases of the bone marrow in which unrestrained proliferation of white blood cells occurs, usually accompanied by anemia, impaired blood clotting, and enlargement of the lymph nodes, liver and spleen. It is believed to that angiogenesis plays a role in the abnormalities in the bone marrow that give rise to leukemia tumors and multiple myeloma diseases.

One of the most frequent angiogenic diseases of childhood is the hemangioma. A hemangioma is a tumor composed of newly-formed blood vessels. In most cases the tumors are benign and regress without intervention. In more severe cases, the tumors progress to large cavernous and infiltrative forms and create clinical complications. Systemic forms of hemangiomas, hemangiomatoses, have a high mortality rate. Therapy-resistant hemangiomas exist that cannot be treated with therapeutics currently in use.

Angiogenesis is also responsible for damage found in heredity diseases such as Osler-Weber-Rendu disease, or heredity hemorrhagic telangiectasia. This is an inherited disease characterized by multiple small angiomas, tumors of blood or lymph vessels. The angiomas are found in the skin and mucous membranes, often accompanied by epitaxis (nose bleeds) or gastrointestinal bleeding and sometimes with pulmonary or hepatitic arteriovenous fistula.

Angiogenesis is also involved in normal physiological processes, such as reproduction and wound healing. Angiogenesis is an important step in ovulation and also in implantation of the blastula after fertilization. Prevention of angiogenesis could be used to induce amenorrhea, to block ovulation, or to prevent implantation by the blastula.

In wound healing, excessive repair or fibroplasia can be a detrimental side effect of surgical procedures and may be caused or exacerbated by angiogenesis. Adhesions are a frequent complication of surgery and lead to problems such as small bowel obstruction.

Several compounds have been used to inhibit angiogenesis. Taylor, et al. (Nature, 297:307 (1982)) have used protamine to inhibit angiogenesis. The toxicity of protamine limits its practical use as a therapeutic. Folkman, et al. (Science, 221:719 (1983), and U.S. Pat. Nos. 5,001,116 and 4,994,443) have disclosed the use of heparin and steroids to control angiogenesis. Steroids, such as tetrahydrocortisol, which lack glucocorticoid and mineralocorticoid activity, have been found to be angiogenic inhibitors.

Other factors found endogenously in animals, such as a 4 kDa glycoprotein from bovine vitreous humor and a cartilage derived factor, have been used to inhibit angiogenesis. Cellular factors, such as interferon, inhibit angiogenesis. For example, interferon alpha or human interferon beta have been shown to inhibit tumor-induced angiogenesis in mouse dermis stimulated by human neoplastic cells. Interferon beta is also a potent inhibitor of angiogenesis induced by allogeneic spleen cells. (Sidky, et al., Cancer Res., 47:5155-61(1987)). Human recombinant interferon (alpha/A) was reported to be successfully used in the treatment of pulmonary hemangiomatosis, an angiogenesis-induced disease. (White, et al., New Eng. J. Med., 320:1197-1200 (1989)).

Other agents which have been used to inhibit angiogenesis include ascorbic acid ethers and related compounds. (Japanese Kokai Tokkyo Koho No.58-13 (1978)). Sulfated polysaccharide DS 4152 also inhibits angiogenesis. (Japanese Kokai Tokkyo Koho No. 63-119500). Additional anti-angiogenic compounds include Angiostatin® (U.S. Pat. Nos. 5,639,725; 5,792,845; 5,885,795; 5,733,876; 5,776,704; 5,837,682; 5,861,372, and 5,854,221) and Endostatin™ (U.S. Pat. No. 5,854,205).

Another compound which has been shown to inhibit angiogenesis is thalidomide. (D'Amato, et al., Proc. Natl. Acad. Sci., 90:4082-85 (1994)). Thalidomide is a hypnosedative that has been successfully used to treat a number of angiogenesis-associated diseases, such as rheumatoid arthritis (Gutierrez-Rodriguez, Arthritis Rheum., 27 (10):1118-21 (1984); Gutierrez-Rodriguez, et al., J. Rheumatol., 16(2):158-63 (1989)), Behcet's disease (Handley, et al., Br. J. Dermatol., 127 Suppl, 40:67-8 (1992); Gunzler, Med. Hypotheses, 30(2):105-9 (1989)), graft versus host rejection (Field, et al., Nature, 211(55): 1308-10 (1966); Heney, et al., Br. J. Haematol., 78 (1):23-7 (1991)), Mycobacteria diseases (Vicente, et al., Arch. Intern. Med., 153(4):534 (1993)), Herpes simplex and Herpes zoster infections (Naafs, et al., Int. J. Dermatol., 24(2):131-4 (1985)), chronic inflammation, ulcerative colitis (Meza, et al., Drug Ther, 23 (11): 74-80, 83 (1993); Powell, et al., Br. J. Dermatol., 113 Suppl 28: 141-4 (1985)), leprosy (Barnes, et al., Infect. Immun., 60(4):1441-46 (1992)) and lupus (Burrows, BMJ, 307: 939-40 (1993)).

Although thalidomide has minimal side effects in adults, it is a potent teratogen. Thus, there are concerns regarding its use in women of child-bearing age. Although minimal, there are a number of side effects which limit the desirability of thalidomide as a treatment. One such side effect is drowsiness. In a number of therapeutic studies, the initial dosage of thalidomide had to be reduced because patients became lethargic and had difficulty functioning normally. Another side effect limiting the use of thalidomide is peripheral neuropathy, in which individuals suffer from numbness and disfunction in their extremities.

Another compound which has been shown to inhibit angiogenesis is 2-methoxyestradiol (2ME₂). 2-Methoxyestradiol is an endogenous, steroidal metabolite of estradiol (E₂) with no intrinsic estrogenic activity, that has potent anti-proliferative activity, and induces apoptosis in a wide variety of tumor and non-tumor cell lines. When administered orally, it exhibits anti-tumor, anti-proliferative, and antiangiogenic activity with little or no toxicity. In vitro data suggests that 2-methoxyestradiol does not engage the estrogen receptor for its anti-proliferative activity and is not estrogenic over a wide range of concentrations, as assayed by estrogen dependent MCF-7 cell proliferation. Currently, 2ME₂ is in several phase-I and II clinical trials under the name PANZEM™.

Therefore, what is needed are new compounds, compositions, and methods which can inhibit angiogenesis. What is also needed is a composition and method which can inhibit the unwanted growth of blood vessels, especially in tumors. A composition and method for antiproliferative activity with respect to endothelial cell growth would be desirable. Further, compounds, compositions, and methods for treating disease states characterized by abnormal cell mitosis or abnormal proliferative activity, or by any combination of angiogenesis, abnormal cell mitosis or abnormal proliferative activity would be useful.

Improved methods and compositions are also needed that are easily administered and capable of inhibiting angiogenesis and exhibiting endothelial cell antiproliferative activity. In addition, what is also desirable are safe and effective treatments that do not create unwanted side effects. Further, a series of compounds that constitute analogs of 2-methoxyestradiol which are non-steroidal in structure and which will have similar biological properties to 2-methoxyestradiol and that can be used in similar applications would be useful.

### SUMMARY OF THE INVENTION

The present invention comprises compounds, compositions, and methods for treating disease states characterized by abnormal angiogenesis, abnormal proliferative activity, abnormal cell mitosis, or a combination of these events. More particularly, the present invention is directed to non-steroidal analogs of 2-methoxyestradiol (2ME₂), including their salts and metabolites, pharmaceutical compositions of non-steroidal analogs of 2ME₂, including their salts and metabolites, and the use of non-steroidal analogs of 2ME₂, including their salts and metabolites, to treat diseases sensitive to such analogs, salts, and metabolites. In another aspect of this invention, non-steroidal analogs of 2ME₂, and salts and metabolites thereof, are used to treat diseases characterized by abnormal cell mitosis and/or abnormal angiogenesis and/or abnormal proliferative activity, including their effect on tumors.

Specifically the present invention relates to non-steroidal analogs of 2-methoxyestradiol, and salts and metabolites thereof. This invention comprises compounds, compositions, and methods related to chemical species of general formulae that inhibit cell proliferation. This invention also comprises compounds within the general formulae that exhibit antitumor activity and compounds within the general formulae that inhibit angiogenesis. Typically, the compounds and compositions of this invention may also exhibit a change (increase or decrease) in estrogen receptor binding, improved absorption, transport (e.g. through blood-brain barrier and cellular membranes), biological stability, or decreased toxicity. The invention also comprises compounds useful in the method, as described by the general formulae of the claims.

Steroids are a general class of organic molecules containing four rings (three six-membered (cyclohexyl or aryl) rings and one cyclopentyl ring) having the general structure in FIG. 1. The rings are generally labeled A, B, C and D. 2-Methoxyestradiol has an aromatic A ring and a methoxy substituent at position 2 and alcohols at positions 3 and 17. Structure activity relationships of estradiol analogs have been reported and have demonstrated that substituents other than methoxy (such as propyne, ethoxy and propene) at position 2 have potent in vitro antiproliferative activity (Cushman et al J. Med. Chem. 1995, 38, 2041).

A novel series of compounds are discovered and proposed that retain the biological activities of 2ME₂. Further, such compounds in accordance with the present invention are expected to have varying, including reduced, metabolism. Contrary to what is observed with 2ME₂, these new analogs are expected to have selective in vitro antiproliferative activity for the endothelial cells over the tumor cell lines to be assessed.

In the present invention, analogs of 2-methoxyestradiol lacking portions of the four ring substructures are proposed to have similar biological activity to 2-methoxyestradiol. As indicated below, these analogs are structurally related to the 2-methoxyestradiol ring system, i.e., such analogs are structural fragments of 2-methoxyestradiol, but will not have the complete steroidal backbone as shown in FIG. 1. It is noted that rings that are shown in FIG. 1 as 6-member rings can also be 4, 5, or 7-member rings and may be saturated or unsaturated, and the ring shown as a five-member ring may also be a 4, 6, or 7-member ring and may be saturated or unsaturated.

The present invention comprises non-steroidal analogs of 2ME₂ of the following general formulas:

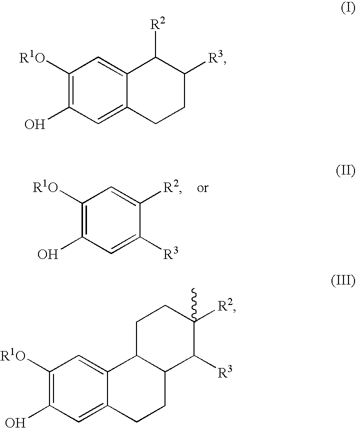

wherein R¹ is independently selected from an alkyl, aryl, substituted alkyl or substituted aryl, any one of which having up to 13 carbon atoms; and

wherein R² and R³ are independently selected from

hydrogen;

halogen, selected from F, Cl, Br, or I;

substituted or unsubstituted alkyl, alkenyl, alkynyl, aromatic group, heterocyclic group, aryl, aralkyl, ether, amine, acyl, formyl, alkoxide, aryloxide, phosphate, trifluoroalkyl, thiol, alkyl thiol, aryl thiol, carboxylic acid, sulfonic acid, amino, alkyl amino, dialkyl amino, ester, cyano, sulfate, sulfonate, sulfone, sulfamate, imine, amide, alkyl amide, or dialkyl amide, any one of which having up to 13 carbon atoms;

[NH₃]⁺X⁻, where X is selected from F, Cl, Br, or I;

a noncyclic heteroatom-containing group with up to 13 carbon atoms, wherein the heteroatom is selected from Si, N, P, O, or S;

wherein any substituted group comprises substituents selected from OH, F, Cl, Br, I, NH₂, OH, SH, OR, SiHₙR₃₋ₙ, where n is an integer from 1-3 inclusive, NHR, NR₂, SR, or PR₂, where R is independently selected from an alkyl or aryl, either of which having up to 10 carbon atoms;

or a metabolite or a salt thereof.

In the general formulas presented here, typically R¹ is selected from hydrocarbyl having from 1 to 13 carbon atoms; R² and R³ are independently selected from H, hydrocarbyl containing from 1 to 13 carbon atoms, or substituted hydrocarbyl containing from 1 to 12 carbon atoms, and wherein when R² is substituted hydrocarbyl, R² may be substituted with halide, OH, NH₂, and the like. Thus, a compound according to the general formulas may have R²═H or R³═H, even simultaneously. Further, any carbon atom in the R² or R³ group may substituted with a heteroatom, such as Si, O, S, N, P, halogen-containing group, and the like, in which each heteroatom is bonded to the appropriate number of hydrogen atoms or hydrocarbyl groups to satisfy its valence. In FIGS. 2-4, the R group which comprises a substitutent on R² or R³ is typically selected from H, F, Cl, Br, I, NH₂, OH, ═CH₂, or ═CHCH₃, and may also include any number of substituents as disclosed herein, including known biological isosteres such as amines, amides, and the like.

Compounds of the present invention also comprise the species shown in FIGS. 2 and 3, but the present invention is not limited to these compounds. Although the examples illustrated in the figures are exclusively carbon chains, it is also an aspect of the present invention that heteroatoms, such as Si, O, S, N, P, and the like, may be substituted for carbon without loss of the anti-angiogenic properties of these molecules. In all cases, it is understood by one of ordinary skill that appropriate substitutions may be made to all atoms such that they satisfy the appropriate valence. Similarly, although most of the carbon substituents are indicated as being hydrogen, some or all of these hydrogens can be replaced by more-polar moieties, including but not limited to, fluorine or other halides, hydroxyl, ester, amino, or alkylamine substituents which increase solubility and/or reduce metabolism and/or improve ADMET (absorption, disposition, metabolism, excretion, or toxicology) characteristics. The substituents on the unsaturated ring, which are positionally equivalent to the 2 and 3 positions of 2-methoxyestradiol and which are shown in the figures as their typical embodiments as methoxy and hydroxyl groups, can be replaced by groups which include, but not limited to, halides, other alkoxy groups, propyne or other alkenes or alkynes, carboxyl or ester groups, and amines or other alkylated amino or amido groups.

Other features and advantages of the invention will be apparent from the following description of preferred embodiments thereof.

### BRIEF DESCRIPTION OF THE FIGURES

FIG. 1 illustrates a general structure of a steroid, in which rings are specified by A, B, C, or D and also provides the structure of 2-methoxyestradiol (2ME₂).

FIG. 2 provides some examples of compounds encompassed by the present invention. Typically, R is independently selected from H, OH, ═CH₂, or ═CHCH₃.

FIG. 3 provides some examples of compounds encompassed by the present invention. Typically, R is independently selected from H, OH, ═CH₂, or ═CHCH₃.

FIG. 4 provides some examples of compounds encompassed by the present invention. Typically, R is independently selected from H, OH, ═CH₂, or ═CHCH₃.

### DETAILED DESCRIPTION OF THE INVENTION

As described herein, compounds that are useful in accordance with the present invention include novel non-steroidal analogs of 2-methoxyestradiol, including salts and metabolites thereof, that exhibit either anti-angiogenic, anti-proliferative, or anti-tumor properties, or any combination of such properties. Further, typical compounds of the present invention are those analogs of 2-methoxyestradiol (2ME₂) in which only a portion of the tetracyclic ring structure is intact.

The non-steroidal compounds of the present invention include compounds of the formula:

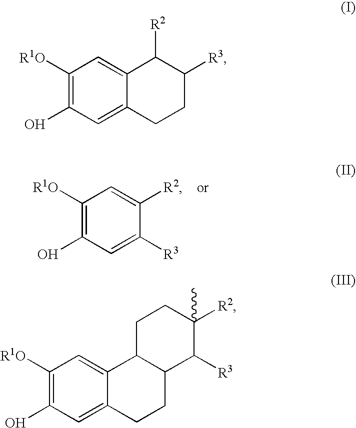

wherein R¹ is independently selected from an alkyl, aryl, substituted alkyl or substituted aryl, each of which having up to 13 carbon atoms; and

wherein R² and R³ are independently selected from

hydrogen;

halogen, selected from F, Cl, Br, or I;

substituted or unsubstituted alkyl, alkenyl, alkynyl, aromatic group, heterocyclic group, aryl, aralkyl, ether, amine, acyl, formyl, alkoxide, aryloxide, phosphate, trifluoroalkyl, thiol, alkyl thiol, aryl thiol, carboxylic acid, sulfonic acid, amino, alkyl amino, dialkyl amino, ester, cyano, sulfate, sulfonate, sulfone, sulfamate, imine, amide, alkyl amide, or dialkyl amide, each of which having up to 13 carbon atoms;

[NH₃]⁺X⁻, where X is selected from F, Cl, Br, or I;

a noncyclic heteroatom-containing group with up to 13 carbon atoms, wherein the heteroatom is selected from Si, N, P, O, or S;

wherein any substituted group comprises substituents selected from OH, F, Cl, Br, I, NH₂, OH, SH, OR, SiHₙR₃₋ₙ, where n is an integer from 1-3 inclusive, NHR, NR₂, SR, or PR₂, where R is independently selected from an alkyl or aryl, either of which having up to 10 carbon atoms;

or a metabolite or a salt thereof.

For example, this invention comprises compounds of the general structures (I), (I), and (III) shown above, wherein only one of R² or R³ in structures (I) (II), and (III) is hydrogen. This invention also comprises compounds of the general structures (I), (I), and (III) shown above, wherein none of R² or R³ in structures (I), (II), and (III) is hydrogen.

Further, this invention comprises compounds of the general structures (I), (I), and (III) wherein:

R¹ is independently selected from an alkyl, aryl, substituted alkyl or substituted aryl, each of which having up to 13 carbon atoms; and wherein

R² and R³ are independently selected from hydrogen; substituted or unsubstituted alkyl, alkenyl, alkynyl, aromatic group, heterocyclic group, aryl, aralkyl, each of which having up to 13 carbon atoms; or a noncyclic heteroatom-containing group having up to 13 carbon atoms, wherein the heteroatom is selected from Si, N, P, O, or S;

or a metabolite or a salt of these compounds.

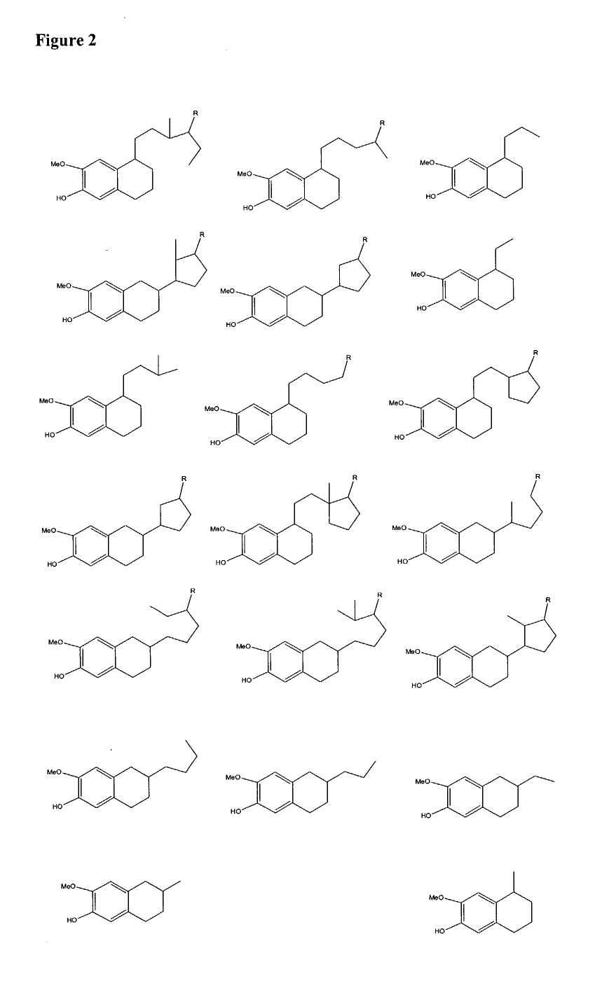

Further, this invention encompasses the compounds shown in FIGS. 2-4. As described herein, the compounds of this invention may be prepared according to the reaction schemes and examples presented herein.

Compounds comprising these structures include, but are not limited to, the structures shown in FIGS. 2, 3, and 4. In FIGS. 2-4, the R group which comprises a substitutent on R² or R³ is typically selected from H, F, Cl, Br, I, NH₂, OH, ═CH₂, or ═CHCH₃, and also may include any number of substituents as disclosed herein, including known biological isosteres such as amines, amides, and the like. Thus, compounds according to this general formula may have R²═H or R³═H, even simultaneously.

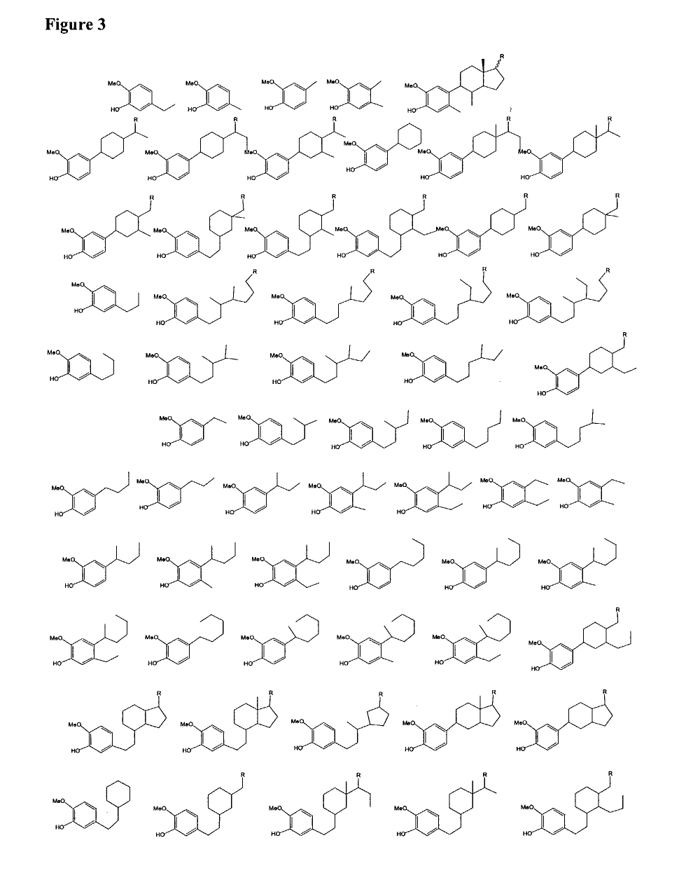

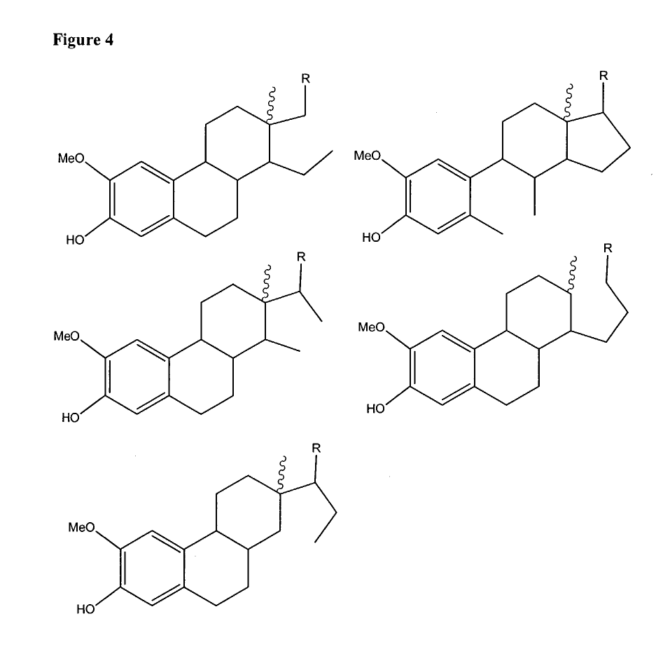

In general terms, the analogs of this invention have only a portion of the steroidal tetracyclic ring structure retained or intact. The analogs shown in the figures may be modified in any regiochemical position, where it is chemically possible, at either or both the A or B rings in FIG. 2, or the A (phenyl) ring in FIG. 3, or the A, B, or C rings in FIG. 4. Further, the methoxy (OMe) and the hydroxy (OH) substituents shown in the structures of FIGS. 2, 3, or 4 may also be substituted with hydrogen, as well as any halogen-containing group, C—, N—, O—, S—, P—, Si—, or other groups as indicated herein. Moreover, it is not necessary that these substituents be limited to the regioisomers shown, as various substitution patterns around the A, B, or C rings shown in FIGS. 2, 3, or 4 are possible, without loss of anti-angiogenic and/or anti-proliferative and/or anti-tumor activity.

Combinations which are physically impossible are not contemplated by this invention, such as a carbon atom containing 5 bonds. The various substituted positions of any of the ring structures shown in FIGS. 2-4, and generically shown in the claims, including the methoxy and hydroxy groups of FIGS. 2-4, may be modified with any of the following groups:

a) alkyls, including both straight and branched alkyls having up to ten carbon atoms and having either alpha or beta stereochemistry, and which may be saturated or unsaturated, substituted or unsubstituted);

b) alkenyls, including, but not limited to, olefin regio- and/or stereoisomers, such as E- and Z-configurations of the olefin, and the hydrocarbon chain of the alkenyl can be straight or branched, have up to ten carbon atoms, may be saturated or unsaturated, substituted or unsubstituted, and have the C═C at any position;

c) alkynyls, which include either straight or branched alkyl chains, have up to ten carbon atoms, may be saturated or unsaturated, substituted or unsubstituted, and have the C≡C at any position;

d) any of the above alkyls, alkenyls, and alkynyls having aromatic or hetero groups incorporated into the respective chains thereof, either singly or in combinations thereof, and wherein the aromatic groups include, but are not limited to, phenyl, phenol, aniline, anisole, toluene (ortho, meta or para derivatives), xylenes, and the hetero groups include, but are not limited to, ether, amine, carbonyl containing functional groups, alcohols, phosphates, trifluoro and thiol groups, acids, esters, sulfates, sulfonates, sulfones, sulfamates and amides;

e) mono, dialkyl or trialkyl amine substitutions with either the alpha or beta stereochemistry, wherein the alkyl can be either straight or branched and have up to ten carbon atoms;

f) —CF₂, —CHF₂, —CF₃ and longer carbon chains having up to 13 carbon atoms, such as trifluoroethanes, pentafluoroethanes, fluorinated alkyl or alkene chains having up to ten carbon atoms, with the position on the chain varying with what is chemically possible to one of skill in the art;

g) hetero groups other than those of d) and e) that are not substituted, mono-substituted or multiply substituted;

h) aromatic groups other than those of d) that are not substituted, mono-substituted or multiply-substituted;

i) both an alkyl group and a hetero or aromatic group incorporated at a single position simultaneously; and

j) geminal alkyl, hetero, or aromatic groups incorporated simultaneously (geminal is defined as two substituents at the same carbon atom).

A hetero group is defined herein as any group which contains at least one atom that is not C or H. A hetero group may contain other substituents, such as aromatic rings and other functional groups. The hetero group may be directly attached to the ring or on a substituent of a group. Especially considered are O, N, S, and P.

In addition, substantially pure isomers of the compounds of the present invention, including up to 100% pure isomers thereof, are contemplated by this invention, however a stereochemical isomer labeled as α or β may be a mixture of both in any ratio, where it is chemically possible by one skilled in the art.

Particularly considered at substituted positions on the ring structures of the compounds of the present invention are the modifications of acid, amide, amine, linear and branched chain alkanes, alkenes and alkynes with heteroatom substitutions, including, but not limited to: carbonyl, —CO—, —S—, —NH— and/or —O— instead of CH₂ and also optionally substituted with hydroxyl, amino, sulfhydryl, azide, halides, nitro, azides, nitrile, sulfamate, carbamate, phosphate, azos, ester, ether, halide, formamide, nitro, nitrile, sulfide, sulfoxide, sulfate, sulfamate, phosphate, and phosphonate instead of H; single or multiple homocyclic or heterocyclic rings of 3, 4, 5, 6, 7, or 8 members, either saturated or unsaturated, attached directly to the ring positions or linked via linear or branched chain alkanes, alkenes or alkynes with heteroatom substitutions, including, but not limited to, —S—, —NH—, and/or —O—, the ring hydrogens and linker hydrogens optionally being further substituted with groups including, but not limited to, those disclosed above.

Furthermore, unless specified otherwise, at any position on the non-steroid ring structures, the following groups can be incorporated where it is chemically possible:

i) hydrogen;

ii) alkyl chains, straight and branched with stereoisomers and having up to 13 carbon atoms;

iii) alkene or alkyne derivatives of above alkyl chain with the olefin or alkyne moiety at any position and any configuration on the chain. Also included are multiply unsaturated alkyl chains of any configuration having up to 13 carbon atoms. The alkyl chain can be substituted with a phenyl substitutent and substituted phenyl substiutents. Examples include, but are not limited to, aniline, anisole, toluene, phenol, and the like.

iv) alkyl, alkene or alkyne chains having up to 13 carbon atoms (straight or branched) independently containing either one or multiple ester (R is defined in paragraphs ii and iii above), carboxylic acids, ketone (R is defined in paragraphs i, ii and iii above), aldehyde, alcohols, amine (primary, secondary, tertiary, and quaternary, with independent R as defined in paragraphs i, ii and iii above) nitrile, azide, urea (with R defined in paragraphs i, ii and iii above), oxime (and alkyl oxime) and halides, such as F, Cl, Br, I, and pharmaceutically acceptable salts of the above;

v) amines, such as primary, secondary, tertiary and quaternary amines attached directly to the compound, with R groups independently as defined in paragraphs i, ii and iii above, and pharmaceutically acceptable salts;

vi) ethers and polyethers attached directly to the ring, with from 1 to 13 carbon atoms;

vii) polyamines and polyols attached directly to the compound, with from 1 to 13 carbon atoms;

viii) ring structures as indicated below, also including epoxides, aziridines and episulfide:

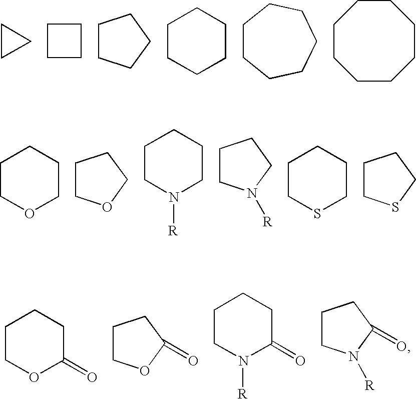

in which the ring structures above may have R groups (defined in parts i-vii and ix-xv) substituted at any position on the ring structure, have varying degrees of unsaturation, and be attached to any position on the compound directly (for example, at a spiro ring junction or at a heteroatom) or through an alkyl or hetero or alkyl hetero chain, and where chemically possible to one skilled in the art;

ix) sulfate, sulfoxide, sulfamate, sulfone, sulfide, disulfide;

x) phosphate, phosphonate;

xi) nitro;

xii) amides substituted with any R group defined in the groups specified in paragraphs i, ii and iii above, attached to the compound through either the carbonyl carbon or amide nitrogen, or linked to the compound by an R group as defined in paragraphs ii and iii above;

xiii) any halogen containing alkyl, alkene and alkyne moiety (for example, CX, CX₂, CX₃ where X═F, Cl, Br, or I);

xiv) —CO(CH₂)ₙOR, where n=0 to 10; the alkyl chain can also contain alkene or alkyne functionalities as defined in i, ii and iii above; and

xv) amino acids or peptides, naturally and unnaturally occurring, having up to 20 amino acids in length.

For example, compounds encompassed by this invention include, but are not limited to, those compounds shown in FIGS. 2-4. In addition, the compounds encompassed by this invention include, but are not limited to, typical compounds such as the following compounds:

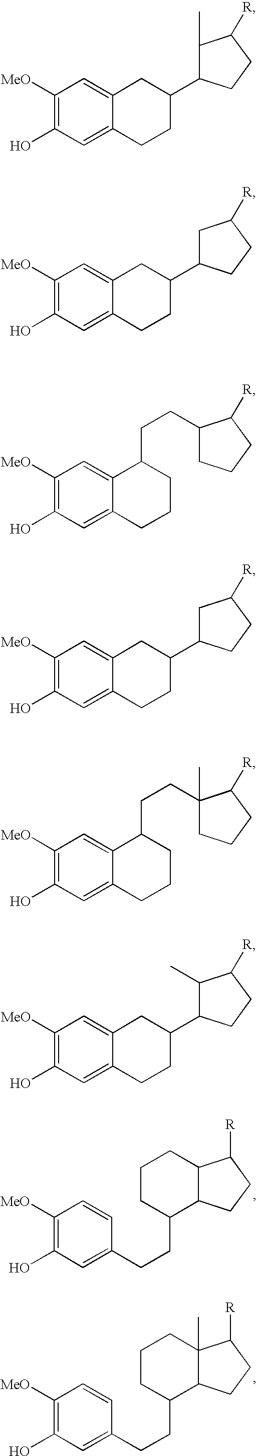

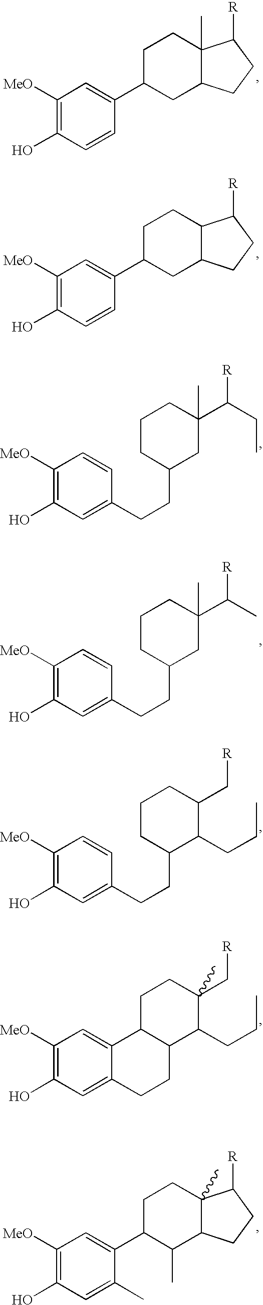

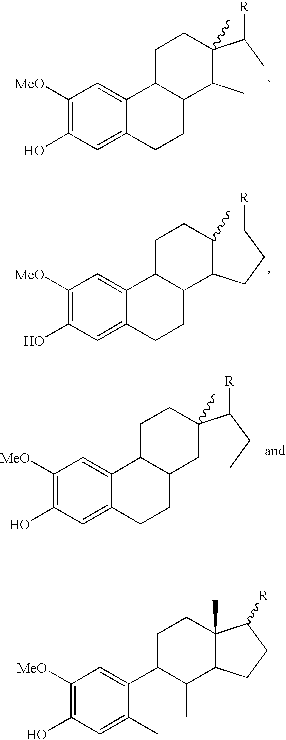

The non-steroidal 2ME₂ analogs of this invention are prepared by a number of synthetic pathways as disclosed herein, and as disclosed in the general reference by Anstead (Anstead, et al. Steroids, 1997, 62, 268), which is incorporated herein by reference. Synthetic schemes used to prepare the compounds of this invention are disclosed in Schemes 1-6, and further disclosed in the Anstead reference.

The present invention also encompasses salts and metabolites of the 2ME₂ analogs disclosed herein. Salts may be formed by any manner known to one skilled in the art, including but not limited to, protonation of an NH₂-substituted or NHR-substituted derivative, and similar methods. Further, this invention comprises metabolites of the compounds disclosed herein, such as may be formed in the course of an animal or human metabolizing any of these species.

The present invention also comprises compounds and pharmaceutical compositions comprising (R)-isomer (at any enantiomeric carbon) of any compound disclosed herein, which are substantially free of the (S)-isomer of that compound. Further, present invention also comprises compounds and pharmaceutical compositions comprising (S)-isomer (at any enantiomeric carbon) of any compound disclosed herein, which are substantially free of the (R)-isomer of that compound. This invention also comprises compounds, and pharmaceutical compositions comprising those compounds, of pure dextrorotatory isomer of any compound disclosed herein, substantially free of the levorotatory isomer of that compound. This invention also comprises compounds, and pharmaceutical compositions comprising those compounds, of pure levorotatory isomer of any compound disclosed herein, substantially free of the dextrorotatory isomer of that compound.

The present invention also comprises compositions, including pharmaceutical compositions, comprising the compounds disclosed herein. This invention further comprises method of using the compounds and compositions of this invention to treat disease states characterized by abnormal cell mitosis, abnormal angiogenesis, abnormal proliferative activity, and by a combination of these events.

Typical compounds of this invention are those analogs of 2-methoxyestradiol (2ME₂) in which only a portion of the tetracyclic ring structure is intact, such as those compounds presented herein. FIGS. 2, 3, and 4 present the structural formulas of typical compounds.

Those skilled in the art will appreciate that the invention extends to other compounds within the formulae given in the claims below, having the described characteristics. These characteristics can be determined for each test compound using the assays detailed below and elsewhere in the literature.

Theoretical Considerations in Mode of Action

2-Methoxyestradiol is an endogenous metabolite of estradiol that has anti-proliferative activity and induces apoptosis in a wide variety of tumor and non-tumor cell lines. When administered orally, it exhibits anti-tumor and anti-proliferative activity with little or no toxicity. It is believed that the non-steroidal analogs of 2-methoxyestradiol will have similar activities as 2ME₂. 2-Methoxyestradiol is metabolized to a less active metabolite, 2-methoxyestrone (2ME₁) as indicated by in vitro and in vivo results. Again, although not wishing to be bound by theory, it is believed that this metabolite is formed through the same enzymatic pathway as estrone is formed from estradiol. Although not wishing to be bound by theory, it is believed that the enzymes responsible for this reaction on estradiol are the 17β-hydroxysteroid dehydrogenases (17β-HSD) which utilize NADP+ as a co-factor (Han et al., J. Biol. Chem. 275:2, 1105-1111 (Jan. 12, 2000) and other references cited earlier). Each of the four members of this enzyme family, types 1, 2, 3, and 4, have distinct activity. It appears that 17β-HSD type 1 catalyzes the reductive reaction (estrone to estradiol), while 17β-HSD type 2 catalyzes the oxidation reaction (estradiol to estrone), and type 3 catalyzes 4-androstenedione to testosterone. It is also believed that an additional metabolic deactivation pathway results in conjugation of 2-methoxyestradiol or 2-methoxyestrone with molecules such as sulfate or glucuronic acid and subsequent loss via excretion. In this invention, non-steroidal 2-methoxyestradiol analogs, including salts and metabolites thereof, may be modified to prevent these metabolic pathways from occurring.

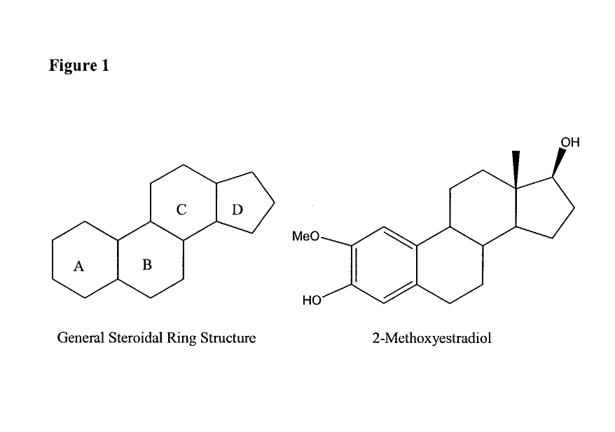

Since 2-methoxyestradiol is metabolized to a much less active metabolite, the present invention modifies the tetracyclic ring structure (see FIG. 1) and its chemical or electrostatic characteristics for retarding or preventing interaction of the family of 17β-hydroxysteroid dehydrogenases and co-factor NADP⁺ on this substrate. This modification of chemical or electrostatic characteristics of 2-methoxyestradiol may also retard or prevent conjugation, such as glucuronidation. It is believed that retardation or prevention of these two metabolic deactivation pathways prolongs the serum lifetime of 2-methoxyestradiol and other estradiol analogs while retaining the desired anti-angiogenic and anti-tumor activity. Assays employed for measuring glucuronidation and conjugation employ substrate enzyme uridine 5′-diphospoglucuronic acid (UDGPA).

It is known that orally-delivered steroids such as estradiol (E₂) and ethynyl-E₂ are extensively metabolized during passage through the gastrointestinal tract and by first-pass metabolism in the liver. Two major metabolic pathways that lead to rapid deactivation and excretion are well studied (Fotsis, T.; Zhang, Y.; Pepper, M. S.; Adlercrcutz, H.; Montesano, R.; Nawreth. P. P.; Schweigerer, L., The Endogenous Estrogen Metabolite 2-Methoxyestradiol Inhibits Angiogenesis and Supresses Tumor. Nature, 1994, 368, 237-239; Wang, Z.; Yang, D.; Mohanakrishnan, A. K.; Fanwick, P. E.; Nampoothiri, P.; Hamel, E.; Cushman, M. “Synthesis of B-Ring Homologated Estradiol Analogs that Modulate Tubulin Polymerization and Microtubule Stability.” J Med. Chem., 2000, 43, 2419-2429) e.g. oxidation at the D-ring's 17-hydroxy group of E₂ to form estrone and conjugation with sulfate and/or glucuronate at the hydroxyls of position-3 on the A-ring and position-17 on the D-ring.

Several studies have been conducted to determine SAR of 2ME₂ analogs (D'Amato, R. J.; Lin, C. M.; Flynn, E.; Folkman, J.; Hamel, E. Inhibition of Angiogenesis and Breast Cancer in Mice by the Microtubule Inhibitors 2-Methoxyestradiol and Taxol”, Cancer Res., 1997, 57, 81-86; Cushman, M.; He, M. -H.; Katzenellenbogen, J. A.; Lin, C. M.; Hamel, E. “Synthesis, Antitubulin and Antimitotic Activity, and Cytotoxicity of Analogs of 2-Methoxyestradiol, an Endogenous Mammalian Metabolite of Estradiol that Inhibits Tubulin Polymerization by Binding to the Colchicine Binding Site.” J Med. Chem. 1995, 38, 2041-2049; and others) but none to reduce or stop its metabolic pathway. Compounds with no chain or with variable methylene chain lengths (1-4) were synthesized by replacing hydroxyl group at position-17 of D-ring of 2ME₂ to block estrone formation or glucuronation. Similarly, several analogs of 17-deoxyestrone with modification at position-2 have been synthesized to block both the glucuronation and hydrolysis of the methoxy group to the hydroxyl. For these analogs data have been presented on the synthesis and preliminary in vitro screening in human umbilical vein endothelial cells (HUVEC) and breast cancer tumor MDA-MB-231 cells for antiproliferative activity, and in MCF-7 tumor cancer cells for estrogenic activity.

Synthesis of Non-Steroidal 2-Methoxyestradiol Analogs

Known compounds that are used in accordance with the invention and precursors to novel compounds according to the invention can be purchased, e.g., from Sigma Chemical Co., St. Louis, Steraloids and Research Plus. Other compounds according to the invention can be synthesized according to known methods from publicly available precursors.

The chemical synthesis of estradiol has been described (Eder, V. et al., Ber 109, 2948 (1976); Oppolzer, D. A. and Roberts, DA. Helv. Chim. Acta. 63, 1703, (1980)). The synthetic pathways used to prepare some of the analogs of the present invention are based on modified published literature procedures for estradiol analogs and dimethylhydrazone (Trembley et al., Bioorganic & Med. Chem. 1995 3, 505-523; Fevig et al., J. Org. Chem., 1987 52, 247-251; Gonzalez et al., Steroids 1982, 40, 171-187; Trembley et al., Synthetic Communications 1995, 25, 2483-2495; Newkome et al., J. Org. Chem. 1966, 31, 677-681; Corey et al Tetrahedron Lett 1976, 3-6; Corey et al., Tetrahedron Lett, 1976, 3667-3668) and German Patent No. 2757157 (1977).

These analogs are prepared by a number of synthetic pathways, a general reference is a Anstead review (Anstead et al Steroids, 1997, 62, 268), which is incorporated herein by reference. It is noted that the Anstead review is a general reference on the SAR of estradiol analogs and their relationship to estrogenic activities. Accordingly, this reference (and references therein) is a general source for synthetic paths for the preparation of 2ME₂ analogs that correspond to the parent estradiol compound.

Scheme 1 provides exemplary methods for preparing the structure (I) compounds of FIG. 2. These compounds are prepared by using the appropriate choices of 6-hydroxy-7-alkoxy-1-tetralone starting compound and alkylating reagent, either the desired alkyl triphenylphosphonium salt or alkyl Grignard according to Scheme 1 (top). Other compounds of FIG. 2 are prepared by using the appropriate choices of starting 6-hydroxy-7-alkoxy-1-tetralone starting compound and alkylating reagent, in this case, the appropriate alkyl halide or other electrophilic alkylating agent, with LDA, according to Scheme 1 (bottom), to provide compounds of a different regiochemistry, as shown in this Scheme and in FIG. 2.

For example, AB ring analogs can be prepared from a α-tetralone precursor as shown in Scheme 1. Asymmetric preparation can be accomplished by use of chiral reagents (such as chiral bases for enolate chemistry or asymmetric hydrogenation catalysts for reductions. Some A-ring analogs can be prepared by nucleophilic addition of the appropriate alkyl Grignard or lithium reagent and subsequent reduction as in Scheme 2.

Schemes 2 and 3 illustrate synthetic approaches to forming the structure (II) compounds such as those shown in FIG. 3. These compounds are prepared by using the appropriate choices of starting 2-alkoxy-4-formyl phenol starting compound and alkylating reagent, either the desired alkyl lithium or alkyl Grignard according to Scheme 2 (top). The other regiochemistry compounds can be prepared similarly, by using the appropriate choices of 2-alkoxy-5-formyl phenol starting compound and alkylating reagent, according to Scheme 2 (bottom). Scheme 3 illustrates another approach to these compounds using the Wolf-Kishner or Clemmensen reduction reactions wherein R is H or alkyl as required.

Scheme 4 provides an illustration of how structure (II) compounds are prepared, in which none of R² or R³ is hydrogen, that is, these compounds are disubstituted structures.

Scheme 5 illustrates another synthetic approach to the compounds of FIG. 3, in which the 6,7-olefinic bond of a steroid compound is cleaved with an oxidation reaction, as indicated, followed by the reduction, deoxygenation, and workup as indicated. This synthetic approach to the compounds of FIG. 3 is particularly useful in allowing the use of a range of substituted steroid compounds to be used as precursors.

Scheme 6 provides an illustration of various bond-cleavage reaction approaches to the structure (III) compounds (P=protecting group) can be employed, using steroid compounds as starting materials. This synthetic approach to the compounds of FIG. 4 is particularly useful in allowing the use of a range of substituted steroid compounds to be used as precursors.

Scheme 7 provides an illustration of a synthetic approach to starting with a steroid core and cleaving the 13,17 bond to form an analog of 2ME₂ with the desired substitution pattern.

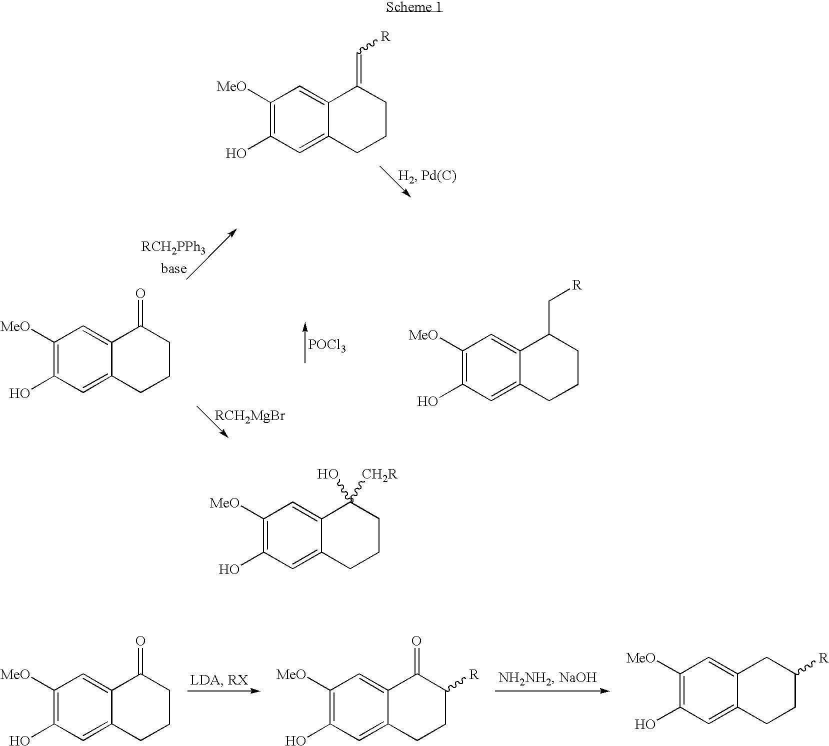

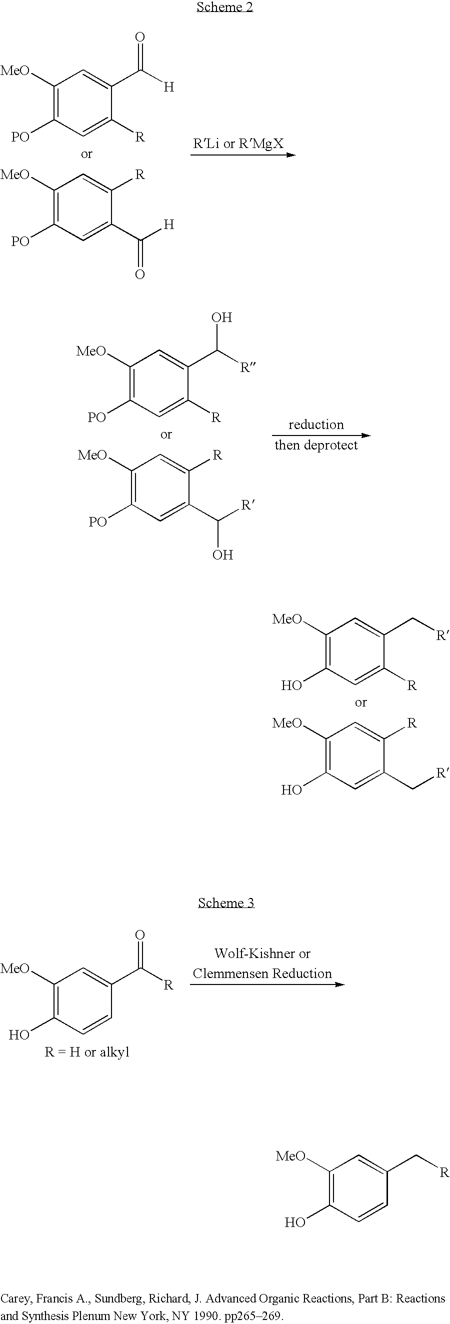

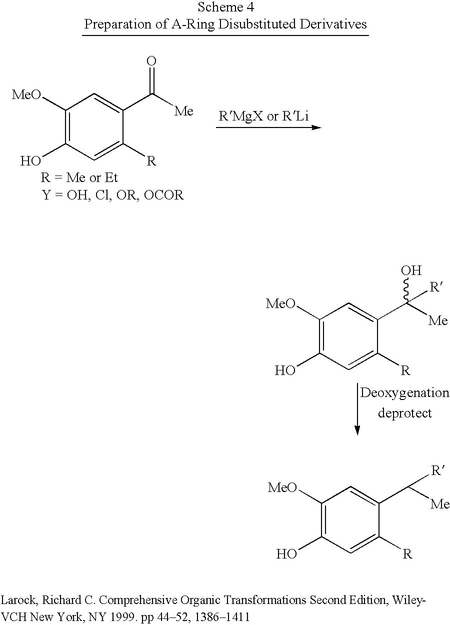

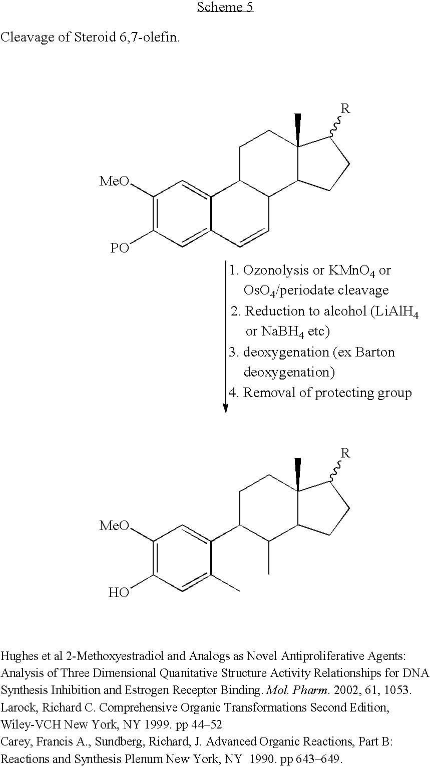

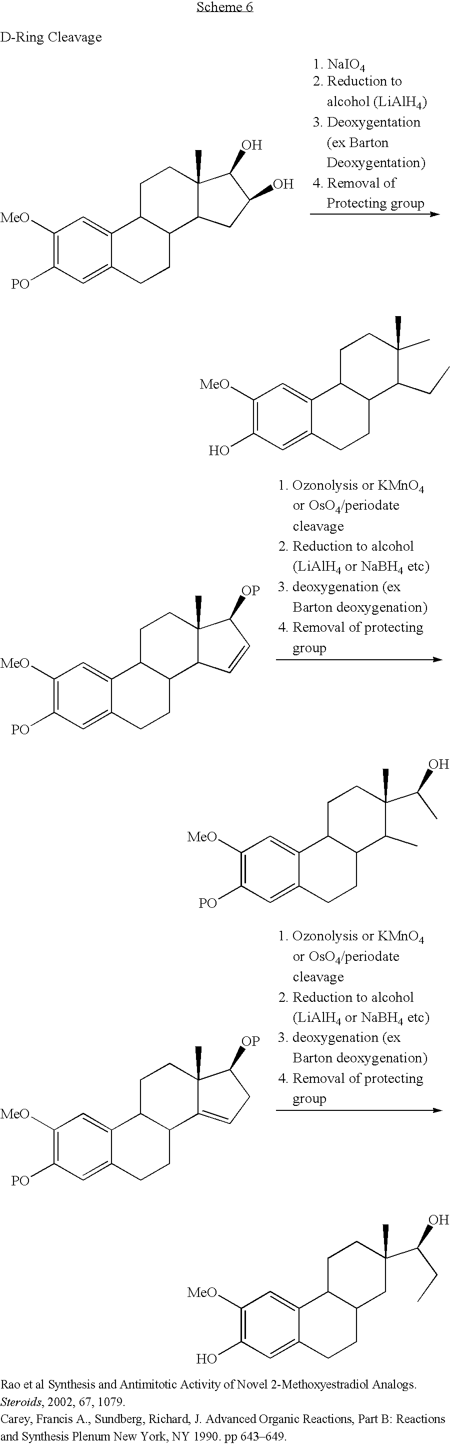

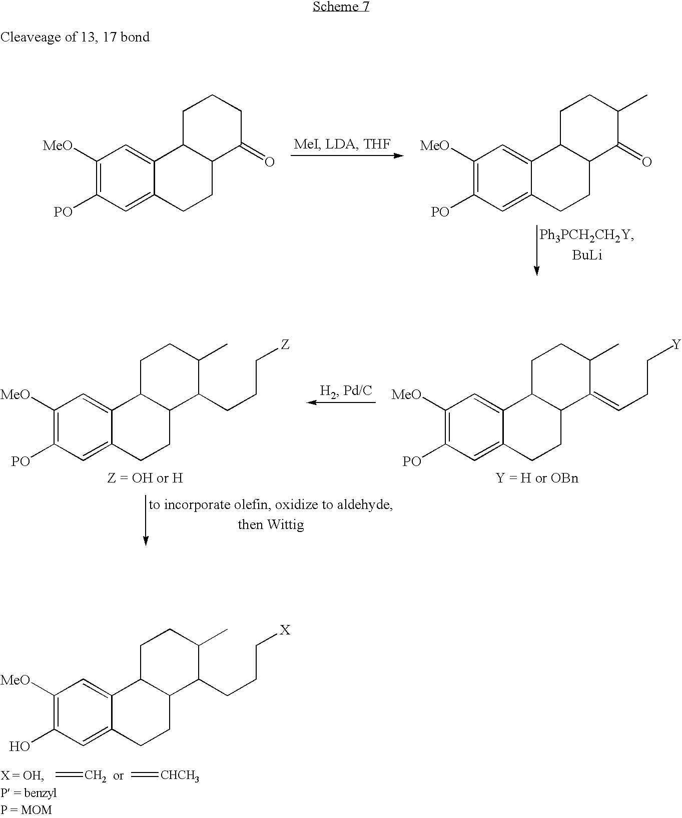

Evaluation of Anti-Proliferative Activity In Situ

Anti-proliferative activity can be evaluated in situ by testing the ability of the new non-steroidal estradiol analogs to inhibit the proliferation of new blood vessel cells (angiogenesis). A suitable assay is the chick embryo chorioallantoic membrane (CAM) assay described by Crum et al. Science 230:1375 (1985). See also, U.S. Pat. No. 5,001,116, hereby incorporated by reference, which describes the CAM assay. Briefly, fertilized chick embryos are removed from their shell on day 3 or 4, and a methylcellulose disc containing the drug is implanted on the chorioallantoic membrane. The embryos are examined 48 hours later and, if a clear avascular zone appears around the methylcellulose disc, the diameter of that zone is measured. Using this assay, a 100 μg disk of the estradiol derivative 2-methoxyestradiol was found to inhibit cell mitosis and the growth of new blood vessels after 48 hours. This result indicates that the anti-mitotic action of 2-methoxyestradiol can inhibit cell mitosis and angiogenesis.

Evaluation of Anti-Proliferative Activity In Vitro

In this invention, analogs of 2-methoxyestradiol which are non-steroidal in structure are proposed to have similar biological properties to 2-methoxyestradiol. The process by which 2ME₂ or its analogs affects cell growth remains unclear, however, a number of studies have implicated various mechanisms of action and cellular targets. 2ME₂ induced changes in the levels and activities of various proteins involved in the progression of the cell cycle. These include cofactors of DNA replication and repair, e.g., proliferating cell nuclear antigen (PCNA) (Klauber, N., Parangi, S., Flynn, E., Hamel, E. and D'Amato, R. J. (1997), Inhibition of angiogenesis and breast cancer in mice by the microtubule inhibitors 2-methoxyestradiol and Taxol., Cancer Research 57, 81-86; Lottering, M -L., de Kock, M., Viljoen, T. C., Grobler, C. J. S. and Seegers, J. C. (1996) 17β-Estradiol metabolites affect some regulators of the MCF-7 cell cycle. Cancer Letters 110, 181-186); Cell division cycle kinases and regulators, e.g., p34^{cdc2} and cyclin B (Lottering et al. (1996); Attalla, H., Mäkelä, T. P., Adlercreutz, H. and Andersson, L. C. (1996) 2-Methoxyestradiol arrests cells in mitosis without depolymerizing tubulin. Biochemical and Biophysical Research Communications 228, 467-473; Zoubine, M. N., Weston, A. P., Johnson, D. C., Campbell, D. R. and Banerjee, S. K. (1999) 2-Methoxyestradiol-induced growth suppression and lethality in estrogen-responsive MCF-7 cells may be mediated by down regulation of p34cdc2 and cyclin B1 expression. Int J Oncol 15, 639-646); transcription factor modulators, e.g., SAPK/JNK (Yue, T -L., Wang, X., Louden, C. S., Gupta, L. S., Pillarisetti, K., Gu, J -L., Hart, T. K., Lysko, P. G. and Feuerstein, G. Z. (1997) 2-Methoxyestradiol, an endogenous estrogen metabolite induces apoptosis in endothelial cells and inhibits angiogenesis: Possible role for stress-activated protein kinase signaling pathway and fas expression. Molecular Pharmacology 51, 951-962; Attalla, H., Westberg, J. A., Andersson, L. C., Aldercreutz, H. and Makela, T. P. (1998) 2-Methoxyestradiol-induced phosphorylation of bc1-2: uncoupling from JNK/SAPK activation. Biochem and Biophys Res Commun 247, 616-619); and regulators of cell arrest and apoptosis, e.g., tubulin (D'Amato, R. J., Lin, C. M., Flynn, E., Folkman, J. and Hamel, E. (1994) 2-Methoxyestradiol, and endogenous mammalian metabolite, inhibits tubulin polymerization by interacting at the colchicine site. Proc. Natl. Acad. Sci. USA 91, 3964-3968; Hamel, E., Lin, C. M., Flynn, E. and D'Amato, R. J. (1996) Interactions of 2-methoxyestradiol, and endogenous mammalian metabolite, with unploymerized tubulin and with tubulin polymers. Biochemistry 35, 1304-1310), p21^{WAF1/CIP1} (Mukhopadhyay, T. and Roth, J. A. (1997) Induction of apoptosis in human lung cancer cells after wild-type p53 activation by methoxyestradiol. Oncogene 14, 379-384), bc1-2 and FAS (Yue et al. (1997); Attalla et al. (1998)), and p53 (Kataoka, M., Schumacher, G., Cristiano, R. J., Atkinson, E. N., Roth, J. A. and Mukhopadhyay, T. (1998) An agent that increases tumor suppressor transgene product coupled with systemic transgene delivery inhibits growth of metastatic lung cancer in vivo. Cancer Res 58, 4761-4765; Mukhopadhyay et al. (1997); Seegers, J. C., Lottering, M -L., Grobler C. J. S., van Papendorp, D. H., Habbersett, R. C., Shou, Y. and Lehnert B. E. (1997) The mammalian metabolite, 2-methoxyestradiol, affects p53 levels and apoptosis induction in transformed cells but not in normal cells. J. Steroid Biochem. Molec.Biol. 62, 253-267). The effects on the level of cAMP, calmodulin activity and protein phosphorylation may also be related to each other. More recently, 2ME₂ was shown to upregulate Death Receptor 5 and caspase 8 in human endothelial and tumor cell lines (LaVallee, T. M., Zhan, X. H., Herbstritt, C. J., Williams, M. S., Hembrough, W. A., Green, S. J., and Pribluda, V. S. 2001. 2-Methoxyestradiol induces apoptosis through activation of the extrinsic pathway. (Manuscript in preparation)). Additionally, 2ME₂ has been shown to interact with superoxide dismutase (SOD) 1 and SOD 2 and to inhibit their enzymatic activities (Huang, P., Feng, L., Oldham, E. A., Keating, M. J., and Plunkett, W. 2000. Superoxide dismutase as a target for the selective killing of cancer cells, Nature. 407:390-5.). All cellular targets described above are not necessarily mutually exclusive to the inhibitory effects of 2ME₂ in actively dividing cells.

The high affinity binding to SHBG has been mechanistically associated to its efficacy in a canine model of prostate cancer, in which signaling by estradiol and 5α-androstan-3α,17β-diol were inhibited by 2ME₂ (Ding, V. D., Moller, D. E., Feeney, W. P., Didolkar, V., Nakhla, A. M., Rhodes, L., Rosner, W. and Smith, R. G. (1998) Sex hormone-binding globulin mediates prostate androgen receptor action via a novel signaling pathway. Endocrinology 139, 213-218).

The more relevant mechanisms described above have been extensively discussed in Victor S. Pribluda, Theresa M. LaVallee and Shawn J. Green, 2-Methoxyestradiol: A novel endogenous chemotherapeutic and antiangiogenic in The New Angiotherapy, Tai-Ping Fan and Robert Auerbach eds., Human Press Publisher.

Assays relevant to the mechanisms of action and cell proliferation are well-known in the art. For example, anti-mitotic activity mediated by effects on tubulin polymerization activity can be evaluated by testing the ability of an estradiol analog to inhibit tubulin polymerization and microtubule assembly in vitro. Microtubule assembly is followed in a Gilford recording spectrophotometer (model 250 or 2400S) equipped with electronic temperature controllers. A reaction mixture typically contains 1.0M monosodium glutamate (pH 6.6), 1.0 mg/ml (10 μM) tubulin, 1.0 mM MgCl₂, 4% (v/v) dimethylsulfoxide and 20-75 μM of a composition to be tested. The reaction mixtures are incubated for 15 min. at 37° C. and then chilled on ice. After addition of 10 μl 2.5 mM GTP, the reaction mixture is transferred to a cuvette at 0° C., and a baseline established. At time zero, the temperature controller of the spectrophotometer is set at 37° C. Microtubule assembly is evaluated by increased turbity at 350 nm. Alternatively, inhibition of microtubule assembly can be followed by transmission electron microscopy as described in Example 2 of U.S. Pat. Nos. 5,504,074, 5,661,143, and 5,892,069.

Other such assays include counting of cells in tissue culture plates or assessment of cell number through metabolic assays or incorporation into DNA of labeled (radiochemically, for example ³H-thymidine, or fluorescently labeled) or immuno-reactive (BrdU) nucleotides. In addition, antiangiogenic activity may be evaluated through endothelial cell migration, endothelial cell tubule formation, or vessel outgrowth in ex-vivo models such as rat aortic rings.

Indications

This invention relates to compounds, compositions, and methods for treating disease states characterized by abnormal angiogenesis, abnormal proliferative activity, and by a combination of these events. Thus, this invention can be used to treat any diseases or disease states characterized by abnormal angiogenesis, abnormal proliferative activity, and by a combination of these events.

Thus, this invention can be used to treat any disease characterized by abnormal cell mitosis. Such diseases include, but are not limited to: abnormal stimulation of endothelial cells (e.g., atherosclerosis), solid tumors and tumor metastasis, benign tumors, for example, hemangiomas, acoustic neuromas, neurofribomas, trachomas, and pyogenic granulomas, vascular malfunctions, abnormal wound healing, inflammatory and immune disorders, Bechet's disease, gout or gouty arthritis, abnormal angiogenesis accompanying: rheumatoid arthritis, skin diseases, such as psoriasis, diabetic retinopathy, and other ocular angiogenic diseases such as retinopathy of prematurity (retrolental fibroplasic), macular degeneration, corneal graft rejection, neuroscular glaucoma, liver diseases and Oster Webber syndrome (Osler-Weber Rendu disease).

This invention can be used to treat any disease characterized by undesired angiogenesis associated with diabetic retinopathy, retinopathy of prematurity, corneal graft rejection; neovascular glaucoma, retrolental fibroplasia; epidemic keratoconjunctivitis; Vitamin A deficiency; contact lens overwear; atopic keratitis; superior limbic keratitis; pterygium keratitis sicca; sjogren's syndrome; acne rosacea; phylectenulosis; syphilis; Mycobacteria infections; lipid degeneration; chemical burns; bacterial ulcers; fungal ulcers; Herpes simplex infections; Herpes zoster infections; protozoan infections; Kaposi's sarcoma; Mooren's ulcer; Terrien's marginal degeneration; marginal keratolysis; trauma; rheumatoid arthritis; systemic lupus; polyarteritis; Wegener's syndrome; sarcoidosis; Scleritis; Stevens-Johnson disease; radial keratotomy; macular degeneration; sickle cell anemia; sarcoid; pseudoxanthoma elasticum; Paget's disease; vein occlusion; artery occlusion; carotid obstructive disease; chronic uveitis; chronic vitritis; Lyme's disease; Eales' disease; Behcet's disease; myopia; optic pits; Stargardt's disease; pars planitis; chronic retinal detachment; hyperviscosity syndromes; toxoplasmosis; post-laser complications; abnormal proliferation of fibrovascular or fibrous tissue; hemangiomas; Osler-Weber-Rendu disease; solid tumors; blood-borne tumors; acquired immune deficiency syndrome; ocular neovascular disease; age-related macular degeneration; osteoarthritis; diseases caused by chronic inflammation; Crohn's disease; ulcerative colitis; tumors of rhabdomyosarcoma; tumors of retinoblastoma; Ewing's sarcoma; with neuroblastoma; tumors of osteosarcoma; leukemia; psoriasis; atherosclerosis; pemphigoid; infections causing retinitis or choroiditis; presumed ocular histoplasmosis; Best's disease; proliferative vitreoretinopathy; Bartonellosis; acoustic neuroma; neurofibroma; trachoma; or pyogenic granulomas.

The compounds, compositions and methods of the present invention may be used to treat a condition selected from an ocular condition, an inflammatory or immune mediated disease, an infectious disease, a cancerous disease, a blood or blood vessel disease, a skin condition, or a tumor in a human or an animal. For example, the ocular conditions, the inflammatory or immune mediated diseases, the infectious diseases, the cancerous diseases, the blood or blood vessel diseases, the skin conditions, or the tumors in a human or an animal include, but are not limited to, an ocular neovascular disease, diabetic retinopathy, retinopathy of prematurity, macular degeneration, corneal graft rejection, neovascular glaucoma, retrolental fibroplasias, epidemic keratoconjunctivitis, due to contact lens overwear, atopic keratitis, superior limbic keratitis, pterygium keratitis sicca, myopia, chronic retinal detachment, optic pits, Terrien's marginal degeneration, hyperviscosity syndromes, chronic uveitis, chronic vitritis, presumed ocular histoplasmosis, retinitis, choroiditis, proliferative vitreoretinopathy, scleritis, Eales' disease, Best's disease, trachoma, due to post-laser complications, Vitamin A deficiency, Sjögren's syndrome, phylectenulosis, lipid degeneration, Kaposi sarcoma, marginal keratolysis, trauma, radial keratotomy, pseudoxanthoma elasticum, Pagets disease, erythematosis, Stargarts disease, pars planitis, a disease associated with rubeosis, a disease associated with Behget's syndrome, rheumatoid arthritis, osterarthritis, ulcerative colitis, Crohn's disease, Mooren's ulcer, arthritis, sarcoidosis, an inflammatory or immune mediated bowel disease, systemic lupus, Wegener's syndrome, Stevens-Johnson disease, pemphigoid, Lyme's disease, acquired immune deficiency syndrome, pyogenic granuloma, gout, gouty arthritis, syphilis, a bacterial infection, a mycobacteria infection, a bacterial ulcer, a fungal ulcer, an Herpes simplex infection, an Herpes zoster infection, a protozoan infection, a Bartonellosis infection, toxoplasmosis, rhabdomyosarcoma, retinoblastoma, Ewing sarcoma, neuroblastoma, osteosarcoma, acoustic neuroma, neurofibroma, hemangioma, a blood borne cancerous disease, vein occlusion, artery occlusion, carotid obstructive disease, polyarteritis, atherosclerosis, Osler-Weber-Rendu disease, sickle cell anemia, leukemia, an acute or chronic neoplastic disease of the bone marrow, a hemangioma, a hereditary hemorrhagic telangiectasia, a disease of the bone marrow, anemia, multiple myeloma, myelo dysplastic syndrome, impaired blood clotting, enlargement of the lymph nodes, liver, or spleen, vascular malfunctions, abnormal wound healing, acne rosacea, due to chemical burns, psoriasis, a blood borne tumor, a cancerous blood borne tumor, a solid tumor, a benign tumor, a cancerous tumor, breast cancer, prostrate cancer, renal cell cancer, a brain tumor, or ovarian cancer.

Another disease which can be treated according to the present invention is rheumatoid arthritis. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Diseases associated with corneal neovascularization that can be treated according to the present invention include but are not limited to, diabetic retinopathy, retinopathy of prematurity, corneal graft rejection, neovascular glaucoma and retrolental ibroplasias, epidemic keratoconjunctivitis, Vitamin A deficiency, contact lens overwear, atopic keratitis, superior limbic keratitis, pterygium keratitis sicca, sjogrens, acne, rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, mariginal keratolysis, trauma, rheumatoid arthritis, systemic lupus, polyarteritis, Wegener's syndrome; sarcoidosis, Scleritis, Steven-Johnson disease, pemphigoid radial keratotomy, and corneal graph rejection.

Diseases associated with retinal/choroidal neovascularization that can be treated according to the present invention include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, trauma and post-laser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy, whether or not associated with diabetes.

Another disease which can be treated according to the present invention is rheumatoid arthritis. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Another disease that can be treated according to the present invention are hemangiomas, Osler-Weber-Rendu disease, or hereditary hemorrhagic telangiectasia, solid or blood borne tumors and acquired immune deficiency syndrome.

Other diseases that can be treated according to the present invention are various metabolic disorders, such as obesity, which is typically associated with abnormal angiogenesis and abnormal proliferative activity.

In addition, the invention can be used to treat a variety of post-menopausal symptoms, osteoporosis, cardiovascular disease, Alzheimer's disease, to reduce the incidence of strokes, and as an alternative to prior estrogen replacement therapies. The compounds of the present invention can work by estrogenic and non-estrogenic biochemical pathways.

This invention also comprises a method of treating a condition selected from an ocular condition, an inflammatory or immune mediated disease, an infectious disease, a cancerous disease, a blood or blood vessel disease, a skin condition, or a tumor in a human or an animal comprising administering to the human or animal a composition comprising a compound as disclosed herein. In addition, this invention further comprises a method of treating these conditions, wherein the composition further comprises an additive selected from an anti-oxidant, a buffer, a bacteriostat, a liquid carrier, an oily solution carrier, a solid carrier, a base, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof. This invention further comprises a method of treating these conditions, wherein the compound is present in the composition in an amount effective upon administration in a daily dose, a daily sub-dose, or any appropriate fraction thereof to the human or animal to reduce the effects of the condition.

Administration

The compositions described above can be provided as physiologically acceptable formulations using known techniques, and these formulations can be administered by standard routes. In general, the combinations may be administered by the topical, oral, rectal or parenteral (e.g., intravenous, subcutaneous or intramuscular) route. In addition, the combinations may be incorporated into biodegradable polymers allowing for sustained release, the polymers being implanted in the vicinity of where delivery is desired, for example, at the site of a tumor or within or near the eye. The biodegradable polymers and their use are described in detail in Brem et al., J. Neurosurg. 74:441-446 (1991) and Examples of bidegradable polymers are discussed in U.S. Pat. No. 5,716,981, both of which are incorporated herein by reference.

The dosage of the composition will depend on the condition being treated, the particular analog used, and other clinical factors such as weight and condition of the patient and the route of administration of the compound. However, for administration to humans, by any means, a dosage of approximately 0.01 to 300 mg/kg/day, wherein approximately 0.05-50 mg/kg/day, is generally sufficient. In further aspect, for administration to humans, by any means, a dosage of approximately 0.1 to 10 mg/kg/day, typically approximately 0.1 to 1 mg/kg/day is sufficient.

The formulations include those suitable for oral, rectal, nasal, topical (including buccal and sublingual), vaginal or parenteral (including subcutaneous, intramuscular, intravenous, intradermal, intraocular, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into associate the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tablets may be made by molding, in a suitable machine, a mixture of the powdered compound moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such as carriers as are known in the art to be appropriate.

Further, the compounds and/or compositions of the present invention can be administered by application of stents. Stents and methods of use thereof are also described in U.S. Pat. No. 5,716,981.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, as herein above recited, or an appropriate fraction thereof, of the administered ingredient.

It should be understood that in addition to the ingredients, particularly mentioned above, the formulations of the present invention may include other agents conventional in the art having regard to the type of formulation in question, for example, those suitable for oral administration may include flavoring agents.

These compounds and formulations comprising these compounds are tested in angiogenesis and anti-tumor assays both in vitro and in vivo. Several in vitro examples are HUVEC, MDA-MB-231 and MCF-7 cell proliferation assays. In vivo examples are B16 melanoma and Lewis Lung metastatic model. Other possible assays are ex vivo systems such as CAM assays and Rat Aortic Ring assays. Structure activity relationships will be examined to determine, e.g. if inversion of any stereocenter results in a change in anti-proliferative activity.

Further evaluation of these compounds can include: in vitro evaluation for antitumor, antiproliferative or antiangiogenic activity using assays such as: in vitro tumor cell line or endothelial cell proliferation assays analyzed by direct cell counts, commercial kits measuring cellular metabolic function including MTT and XTT, or cell counts using metabolic incorporation into DNA of labeled (³H-thymidine) or immunoreactive nucleotide (BrdU); in vitro assay of motility or migration including trans-membrane migration or endothelial cell layer wounding; surrogate in vitro assays for specific functions of 2ME₂ analogs such as tubulin polymerization or SOD or other enzyme binding or inhibition assays; in vitro assays for induction of apoptosis or other perturbation of cell function including TUNEL and histone analysis, oxygen radical levels, p53 levels or p53 phosphorylation, or analysis of levels or activation state of enzymes in the apoptotic pathway such as caspases or other apoptotic molecules such as death receptors or other receptors associated with caspase activation; ex vivo assays including endothelial outgrowth from bone or aortic rings, tube forming assays, mitogenesis or motility or morphogenesis assays; or in vivo assays including chick embryo chorioallantoic membrane assay (CAM), matrigel plug assay, rabbit or mouse corneal eye pocket angiogenesis assay, liver sponge assay, or in vivo assays of angiogenesis-dependent tumor growth including B16BL6 melanoma metastasis or Lewis Lung primary and metastatic rat or mouse models or tumor xenografts or tumor development in susceptible strains such as AJ mice or mutant mouse strains such as agouti or ras-overexpressing strains or the min mouse or other transgenic or mutant mouse model systems. Examples of further analyses which can be used to determine the suitability of these analogs for use in particular diseases and pathologies include: estrogenic activity which can be assessed in vitro using estrogen dependant MCF-7 proliferation assay, or in animal assays such as uterine weight gain or uterine or vaginal cytology or diestrus time perturbation; metabolic stability which can be analyzed using liver microsomes in vitro, or dosing animals or human subjects and measuring metabolism of the compound or formation of specific metabolites such as oxidation or demethylation products or conjugates using analytical techniques including HPLC, LCMS, GCMS, or LCMSMS; models of inflammation-associated angiogenesis including psoriasis, granuloma and collagen-induced arthritis models; the ApoE −/− knockout mouse model of atherosclerotic angiogenesis; porcine model of restenosis injury; neonatal mouse model of hypoxia-driven retinopathy; measurement of cholesterol levels; assays for antiangiogenic effects on fertility or reproduction or endometriosis including inhibition of angiogenesis during follicular development; assays for effect of antiangiogenic agents on wound healing including skin punch biopsy measurement; and osteoporosis models such as in vitro measurement of osteoclast and osteoblast differentiation, proliferation, and function, ex vivo assessment of bone resorption (pitting), or in vivo measurement of bone density.

It should be understood that in addition to the ingredients, particularly mentioned above, the formulations of this invention may include other agents conventional in the art having regard to the type of formulation in question, for example, those suitable for oral administration may include flavoring agents.

In the structures, compounds, compositions, methods and descriptions provided herein, it is to be understood that: saturated bonds in any ring may be dehydrogenated where chemically possible to someone skilled in the art; all stereochemical isomers have either an α or β configuration (R and S; or D- and L-) where chemically possible to someone skilled in the art; lower alkyl is defined as a carbon chain having 1-13 carbon atoms which may be branched or unbranched and wherein chemically possible to one skilled in the art; “terminal” is defined as “at the end of a chain”; the compounds of the present invention may also be presented as a pharmaceutically acceptable salts; and examples of heterogroups that may be used include, but are not limited to, ether groups, amino groups, carbonyl groups, haloalkyl, dihaloalkyl, or trihaloalkyl groups, hydroxy groups, ester groups, dialkylamino, or monoalkylamino groups, thiol, thioether, or thioester (phosphate) groups, and oximes.

References for various syntheses, compounds, structures, compositions, methods and descriptions provided herein, include: Org. Synt. Coll. Vol. 5, 552; Org. Synt. Coll. Vol. 3, 590; and Shah, et. al. J. Med. Chem. 1995, 38, 4284; U.S. Pat. No. 5,504,074; U.S. Pat. No. 5,661,143; U.S. patent application Ser. No. 09/243,158; and U.S. patent application Ser. No. 09/939,208.

As used herein the term “hydrocarbyl” is used to specify a hydrocarbon radical group that includes, but is not limited to aryl, alkyl, cycloalkyl, alkenyl, cycloalkenyl, cycloalkadienyl, alkynyl, aralkyl, aralkenyl, aralkynyl, alkylidene, carbene, and the like, and includes all substituted, unsubstituted, branched, linear, heteroatom substituted derivatives thereof, wherein the hydrocarbyl group has from 1 to about 13 carbon atoms.

Also as used herein, the abbreviations for hydrocarbyl groups, substitutents, and the like, are those normally used. For example, Me is methyl, Et is ethyl, Pr is propyl, Bu is butyl, Ph is phenyl, and so forth.

The term “alkyl” group is used in it usual way, and is used herein to refer to groups having from 1 to about 13 carbon atoms, such as methyl, ethyl, propyl, butyl, pentyl, hexyl, heptyl, octyl, nonyl, decyl, undecyl, dodecyl, and tridecyl. This term encompasses all the structural isomers of a particular alkyl group.

The amino group on the aniline can be substituted with hydrogen, alkyl (C₁-C₁₃, straight chain or branched), cycloalkyl (C₃-C₁₀), or aryl substituted aryl groups. The phenyl ring of this aniline derivative can be optionally substituted with one or more functional groups, or a combination of functional groups such as alkyl, alkenyl, alkynyl, phenyl, benzyl, halo, cyano, nitro, hydroxy, thioxy, alkoxy, aryloxy, haloalkyloxy, alkylthio, arylthio, amino, alkyl amino, aryl amino, acyl, carboxyl, amido, sulfonamido, sulfonyl, sulfate, sulfonic acid, morpholino, piperazinyl, pyridyl, thienyl, furanyl, pyrroyl, pyrazoyl, phosphate, phosphonic acid, or phosphonate. If applicable, these groups can be represented in protected or unprotected forms used in standard organic synthesis.

As used herein and in the appended claims, the singular forms “a,” “an,” and “the” include the plural reference unless the context clearly indicates otherwise. Thus, for example, reference to “a compound” is a reference to one or more such compounds and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are herein described.

All publications and patents mentioned herein, including those presented in the figures and schemes, are incorporated herein by reference for the purpose of describing and disclosing, for example, the constructs and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

For any particular compound disclosed herein, any general structure presented also encompasses all conformational isomers, regioisomers, and stereoisomers that may arise from a particular set of substitutents. The general structure also encompasses all enantiomers, diastereomers, and other optical isomers whether in enantiomeric or racemic forms, as well as mixtures of stereoisomers, as the context requires.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to one of ordinary skill in the art without departing from the spirit of the present invention or the scope of the appended claims.

### EXAMPLE 1

Compounds of the present invention with the following structure are prepared according to this Example, as shown in Scheme 1 (top).

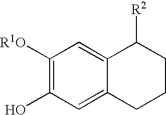

These compounds are prepared by using the appropriate choices of starting 6-hydroxy-7-alkoxy-1-tetralone starting compound and alkylating reagent, either the desired alkyl triphenylphosphonium salt or alkyl Grignard according to Scheme 1 (top).

In this example, the compound shown below, wherein R¹=methyl and R²=methyl, was prepared according to Scheme 1 (top), as follows.

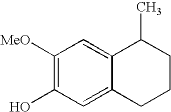

Methyl triphenylphosphonium bromide was dissolved in toluene, and t-amyl potassium alcoholate was added and the resulting mixture was refluxed for 30 min; 6-hydroxy-7-methoxy-1-tetralone was added and refluxed for 4 h. After a standard workup and purification by silica gel chromatography, a 15% yield of the olefin product (Scheme 1) was obtained. This alkene, was reduced using Pd/C (10%) and H₂ gas (at 30 psi) for 2 h, after which the reaction mixture was filtered through celite to remove the catalyst. Following column chromatography purification of the resulting filtrate, a 59% yield was obtained of the desired product shown above (mp 33.5-34.5 ° C.). The ¹H NMR spectrum and elemental analysis of this product were consistent with the structure shown.

Compounds presented in FIG. 2 of the same regiochemistry as this compound are prepared in the same manner, using the required choice of 6-hydroxy-7-alkoxy-1-tetralone starting material and the required alkylating reagent, either an alkyl triphenylphosphonium salt or alkyl Grignard according to Scheme 1 (top).

### EXAMPLE 2

Compounds of the present invention with the following structure are prepared according to this Example, as shown in Scheme 1 (bottom).

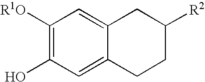

These compounds are prepared by using the appropriate choices of starting 6-hydroxy-7-alkoxy-1-tetralone starting compound and alkylating reagent, in this case, the appropriate alkyl halide or other electrophilic alkylating agent, with LDA, according to Scheme 1 (bottom).

In this example, the compound shown below, wherein R¹=methyl and R²=methyl, was prepared according to Scheme 1 (bottom), as follows.

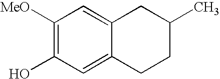

6-tert-Butyldimethylsilylether-7-alkoxy-1-tetralone (other phenolic protected groups besides tBDMS are possible as well) can be dissolved in anhydrous THF, cooled to −78° C. and treated with 1.2 eq LDA and 1.2 eq of the appropriate alkyl halide. The mixture was slowly warmed to rt and monitored by TLC. After the reaction has gone to completion, water is added, and the mixture is extracted with ether. The ether is washed with brine, dried with Na₂SO₄, filtered and solvent can be removed under reduced pressure. Purification by column chromatography is expected to yield the desired ketone. Wolf-Kishner (Li, J. J. Name Reactions, A Collection of Detailed Reaction Mechanisms Springer, New York, N.Y. 2002.) or Clemmensen reduction (Mundy B. P.; Ellard M. G. Name Reactions and Reagents in Organic Synthesis Wiley Interscience, New York, N.Y. 1988.) of the ketone and removal of the silyl ether with TBAF or acid (Greene, T. W.; Wuts, P. G. M. Protective Groups in Organic Synthesis Third Edition, Wiley Interscience, New York, N.Y. 1999.) gives target compound.

Compounds presented in FIG. 2 of the same regiochemistry as this compound are prepared in the same manner, using the required choice of 6-hydroxy-7-alkoxy-1-tetralone starting material and the required electrophilic alkylating reagent, according to Scheme 1 (bottom).

### EXAMPLE 3

Compounds of the present invention with the following structure are prepared according to this Example, as shown in Scheme 2 (top).

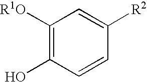

These compounds are prepared by using the appropriate choices of starting 4-hydroxy-3-methoxybenzaldehyde compound and alkylating reagent, either the desired alkyl lithium or alkyl Grignard according to Scheme 2 (top).

In this example, the compound shown below, wherein R¹=methyl and R²=cyclohexane, was prepared according to Scheme 2 (top), as follows.

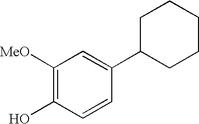

4-hydroxy-3-methoxybenzaldehyde was protected as a MOM ether by dissolving in THF, and diisopropylethylamine the MOMCl was added dropwise. The mixture was heated to 50° C. overnight. The mixture was cooled to room temperature (rt), poured into saturated NH₄Cl, and ether. The layers were separated, and the organic was washed with water then brine. Dry with magnesium sulfate, filtered and solvent was removed under reduced pressure to gime the MOM protected aldehyde. This product was dissolved in THF, and cooled to 0° C. 1.2 eq of the appropriate Grignard or alkyl lithium reagent (in this case either the cyclhexane magnesium bromide or lithium cyclohexane was added dropwise, then the mixture was brought to reflux. The reaction was monitored by TLC and upon completion, the reaction was cooled to rt, and quenched by careful addition of 2N HCl. The mixture was washed with ether, and the ether was washed with brine. Dry with MgSO₄ and remove solvent under reduced pressure to give the desired alcohol. The alcohol was deoxygentated by following the general procedure in Liu et al Tetrahedron Lett. 1985, 4847. The MOM ether was removed by dissolving the MOM ether in THF, and adding 6M HCl. After refluxing for 15 minutes, the reaction was cooled to rt, poured into water and washed with ether. The ether was washed with water, brine, dried with MgSO₄, filtered and solvent was removed under reduced pressure. The product was purified by column chromatography to analytically pure product.

Compounds presented in FIG. 3 of the same regiochemistry as this compound are prepared in the same manner, using the required choice of 2-alkoxy-4-formyl phenol starting material and the required alkylating reagent, according to Scheme 2 (top).

### EXAMPLE 4

Compounds of the present invention with the following structure are prepared according to this Example, as shown in Scheme 2 (bottom).

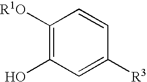

These compounds are prepared by using the appropriate choices of 3-hydroxy-4-methoxybenzaldehyde starting compound and alkylating reagent, either the desired alkyl lithium or alkyl Grignard according to Scheme 2 (bottom).

In this example, the compound shown below, wherein R¹=methyl and R³=CH₂C₆H₁₁, was prepared according to Scheme 2 (bottom)), as follows.

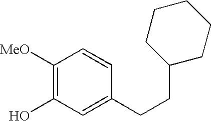

The reaction procedure for Example 3 was followed, except, 3-hydroxy-4-methoxybenzaldehyde was used as starting material and LiCH₂C₆H₁₁ was used as the alkylating reagent.

### EXAMPLE 5

Compounds of the present invention with the following structure are prepared according to this Example, as shown in Scheme 2 (bottom) and the general procedure in Example 3.

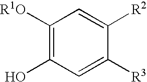

These compounds are prepared by using the appropriate choices of starting 4-hydroxy-5-methoxy-2-methylbenzoic acid or 4-hydroxy-5-methoxy-2-ethylbenzoic acid and alkylating reagent, either the desired alkyl lithium or alkyl Grignard according to Scheme 2.

In this example, the compound shown below, wherein R¹=methyl, R²=ethyl, and R³=methyl, was prepared according to Scheme 3, using 4-Hydroxy-5-methoxy-2-methylbenzaldehyde as starting material and methyl magnesium bromide as the alkylating reagent and the same general procedure as in example 3.

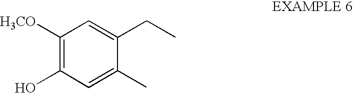

Compounds of the present invention containing branched alkyl substitutions at R₂ such as the example illustrated below can be prepared as in Scheme 4.

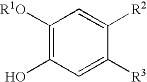

The specific example is illustrated below where R¹=methyl, R²=1′-methyl-propane and R₃=ethyl can be prepared as follows

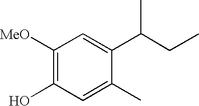

1-(4-hydroxy-5-methoxy-2-methylphenyl)ethanone or 1-(4-hydroxy-5-methoxy-2-ethylphenyl)ethanone can be protected as the MOM ether, followed by alkylation with the appropriate Grignard or alkyl lithium reagent to give the tertiary alcohol and deoxygenated as in scheme 2, example 3.

### EXAMPLE 7

compounds of the present invention where the steroid B-ring is cleaved such as the example illustrated below can be prepared as in scheme (Cleavage of 6,7 olefin)

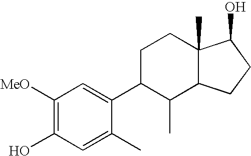

The protected 6,7-olefin derivative of 2ME₂ can be prepared as in Hughes et al Mol Pharm. 2002, 61, 1053. The olefin can be cleaved by a number of methods, including the Lemieux-Johnson type cleavage with sodium periodate and either potassium permanganate or osmium tetroxide. Briefly, the steroid can be dissolved in butanol, and the oxidixing reagents can be dissolve in water, the solutions are mixted, the pH is adjusted to 8-9 with potassium carbonate and the reaction is followed by TLC. After completion of the reaction, the mixture is quenched by addition of HCl, and sodium metabisulfite to periodate, iodate and iodine into iodide. The solution is basified and the butanol is removed under reduced pressure. The remaining solution is acidified, and the product is extracted into ether, dried with sodium sulfate, filtered and solvent is removed under reduced pressure. The resulting carboxylic acid can be reduced using LiAlH₄ deoxygenated and any protecting groups can be removed as in example 3. Alternatively, the olefin can be cleaved by ozonolysis. Ozonolysis is usually done using an ozone generator, and procedures and workups are detailed in several reports including Fieser and Fieser (Reagents for Organic Synthesis Volume 1, Wiley, New York, N.Y. 1967. pp 773-777).

### EXAMPLE 8

Compounds of the present invention where the steroid D-ring is cleaved such as the example illustrated below between carbons 16 and 17 can be prepared as in scheme (D-ring cleavage).

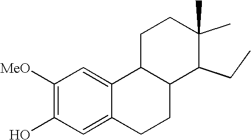

Vic-glycol, 16-Hydroxy-2ME₂ (Nambara et al Chem. Pharm. Bull. 1975, 23, 1613.) can be dissolved in ethanol, and potassium periodate in 1N sulfuric acid is heated at 40° C. Water is added to dissolve the precipitated potassium sulfate and ether extraction will the cleaved bis-aldehyde (Fieser and Fieser Reagents for Organic Synthesis Volume 1. Wiley, New York, N.Y., 1967 pp 815-817). The aldehyde can be reduced, deoxygenated and protecting groups can be removed as in example 3. Lead tetraacetate is an alternate reagent to cleave vic-glycols.

### EXAMPLE 9

Compounds of the present invention where the steroid D-ring is cleaved such as the example illustrated (between carbons 14 and 15 or 15 and 16) below can be prepared as in scheme (D-ring cleavage).

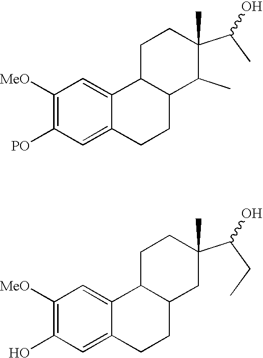

Both the 14,15 and 15,16 olefin precursor can be prepared as in Rao (Steroids 2002, 67, 1079). As in Example 7, either the 14,15- or 15,16-olefin can be cleaved by a number of methods, including the Lemieux-Johnson type cleavage with sodium periodate and either potassium permanganate or osmium tetroxide. Briefly, the steroid can be dissolved in butanol, and the oxidixing reagents can be dissolve in water, the solutions are mixed, the pH is adjusted to 8-9 with potassium carbonate and the reaction is followed by TLC. After completion of the reaction, the mixture is quenched by addition of HCl, and sodium metabisulfite to periodate, iodate and iodine into iodide. The solution is basified and the butanol is removed under reduced pressure. The remaining solution is acidified, and the product is extracted into ether, dried with sodium sulfate, filtered and solvent is removed under reduced pressure. The resulting carboxylic acid can be reduced using LiAlH₄ deoxygenated and any protecting groups can be removed as in Example 3. Alternatively, the olefin can be cleaved by ozonolysis. Ozonolysis is usually done using an ozone generator, and procedures and workups are detailed in several reports including Fieser and Fieser (Reagents for Organic Synthesis Volume 1, Wiley, New York, N.Y. 1967, pp 773-777).

### EXAMPLE 10

Examples where the 13,17 bond is cleaved can be prepared as in Scheme (13,17 cleave—sent today) and an example is illustrated below.

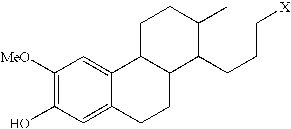

Tricyclic ketone can be alkylated regioselectively using a bulky base such as LDA or LiHMDS to generate the kinetically favored enolate, followed by quenching with methyl iodide at −78° C. Wittig reaction using either the O-protected propane (benzyl ether is an example) or triphenylphosphonium propyl iodide and Butyl lithium will incorporate the propyl side chain in place of the ketone functionality. Catalytic reduction with palladium on carbon and H₂ will simultaneously reduce the olefin and remove the benzyl protecting group if the protected hydroxy group is present. To replace an olefin functionality in place of the hydroxy group, the alcohol can be oxidized to the aldehyde (ex Swern reaction) and Wittig chemistry can convert this carbonyl to an olefin. Removal of the phenolic protecting group completes the sythesis (HCl will memove the MOM protecting group as depicted in the example).

While aspects of the present have been described, it should be understood that various changes, adaptations, and modifications may be made therein without departing form the sprit of the invention and the scope of the appended claims.

We claim:

1. A compound of the formula: 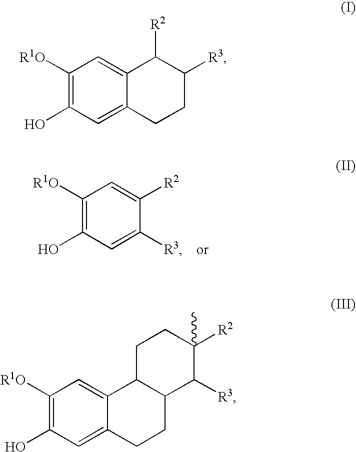
wherein R¹ is independently selected from an alkyl, aryl, substituted alkyl or substituted aryl with up to 13 carbon atoms; and
wherein R² and R³ are independently selected from
hydrogen;
halogen;
substituted or unsubstituted alkyl, alkenyl, alkynyl, aromatic group, heterocyclic group, aryl, aralkyl, ether, amine, acyl, formyl, alkoxide, aryloxide, phosphate, trifluoroalkyl, thiol, alkyl thiol, aryl thiol, carboxylic acid, sulfonic acid, amino, alkyl amino, dialkyl amino, ester, cyano, sulfate, sulfonate, sulfone, sulfamate, imine, amide, alkyl amide, or dialkyl amide, any of which having up to 13 carbon atoms;
[NH₃]⁺X⁻, where X is selected from F, Cl, Br, or I;
a noncyclic heteroatom-containing group with up to 13 carbon atoms, wherein the heteroatom is selected from Si, N, P, O, or S;
wherein any substituted group comprises substituents selected from OH, F, Cl, Br, I, NH₂, OH, SH, OR, SiHₙR₃₋ₙ, where n is an integer from 1-3 inclusive, NHR, NR₂, SR, or PR₂, where R is independently selected from an alkyl or aryl with up to 10 carbon atoms;
or a metabolite or a salt thereof.

2. The compound of claim 1, wherein the compound is

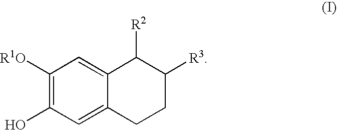

3. The compound of claim 1, wherein the compound is

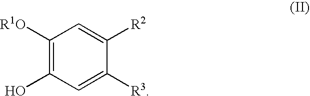

4. The compound of claim 1, wherein the compound is

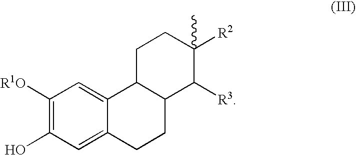

5. The compound of claim 1, wherein only one of R² or R³ in structures (I) (II), and (III) is hydrogen.

6. The compound of claim 1, wherein none of R² or R³ in structures (I), (II), and (III) is hydrogen.

7. The compound of claim 1, wherein
R¹ is independently selected from an alkyl, aryl, substituted alkyl or substituted aryl with up to 13 carbon atoms; and
wherein R² and R³ are independently selected from
hydrogen;
substituted or unsubstituted alkyl, alkenyl, alkynyl, aromatic group, heterocyclic group, aryl, aralkyl, any of which having up to 13 carbon atoms; or
a noncyclic heteroatom-containing group with up to 13 carbon atoms, wherein the heteroatom is selected from Si, N, P, O, or S;
or a metabolite or a salt thereof.

8. The compound of claim 1, wherein the compound is selected from

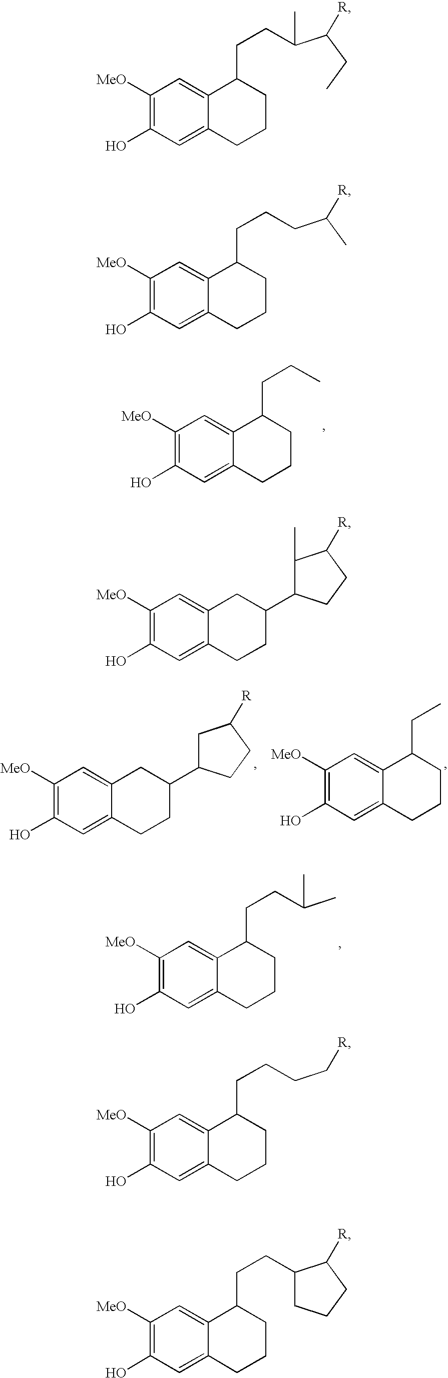

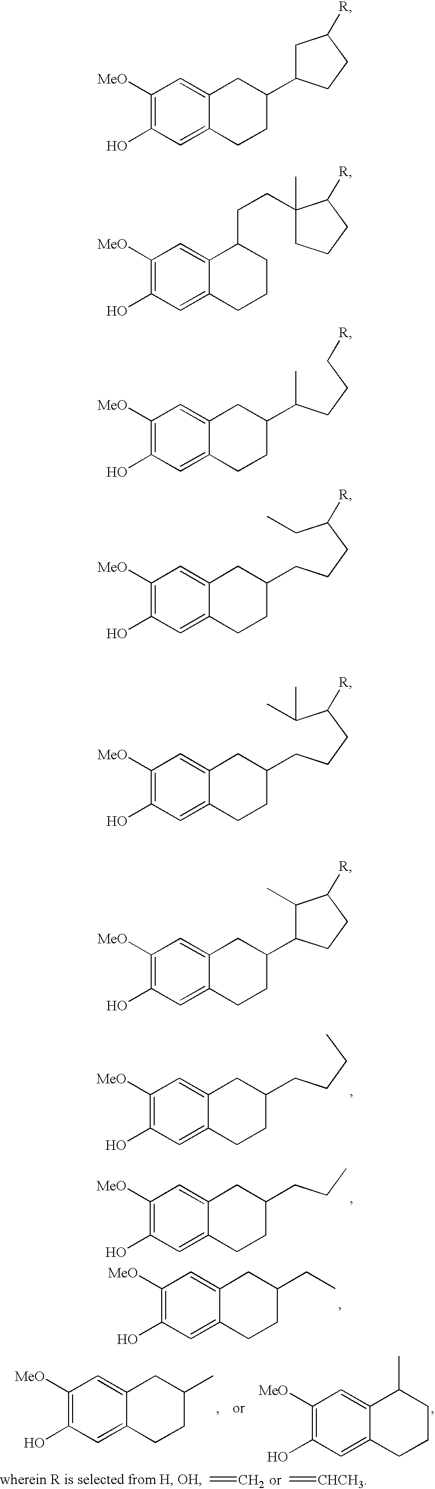

9. The compound of claim 1, wherein the compound is selected from 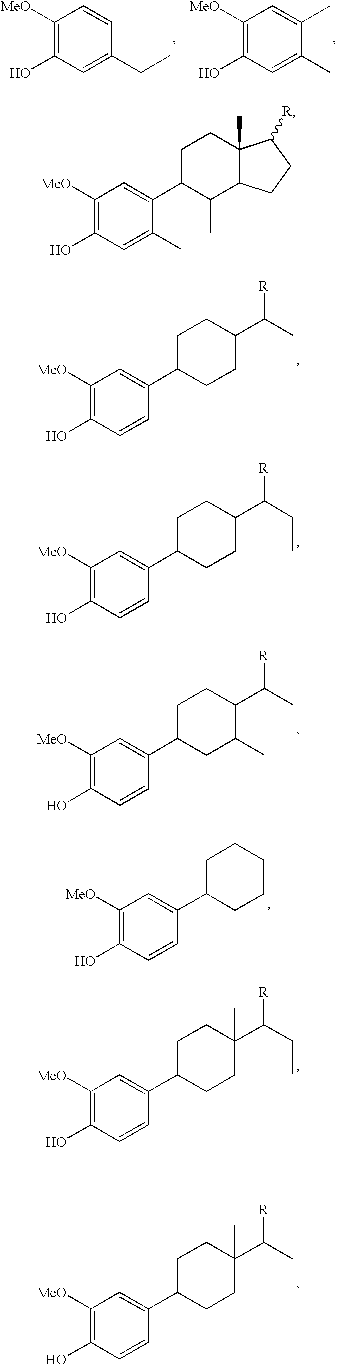 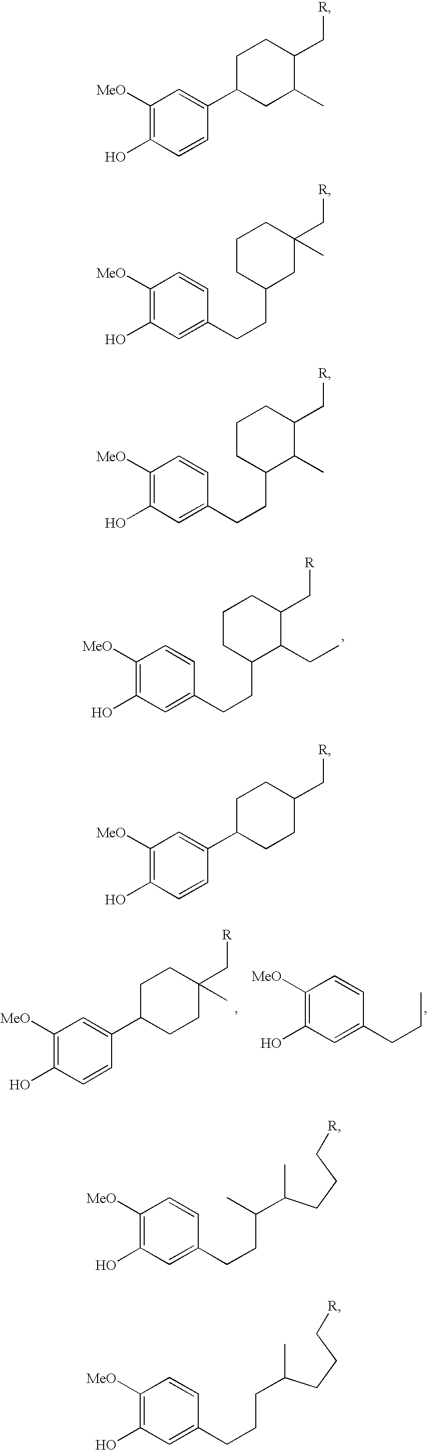 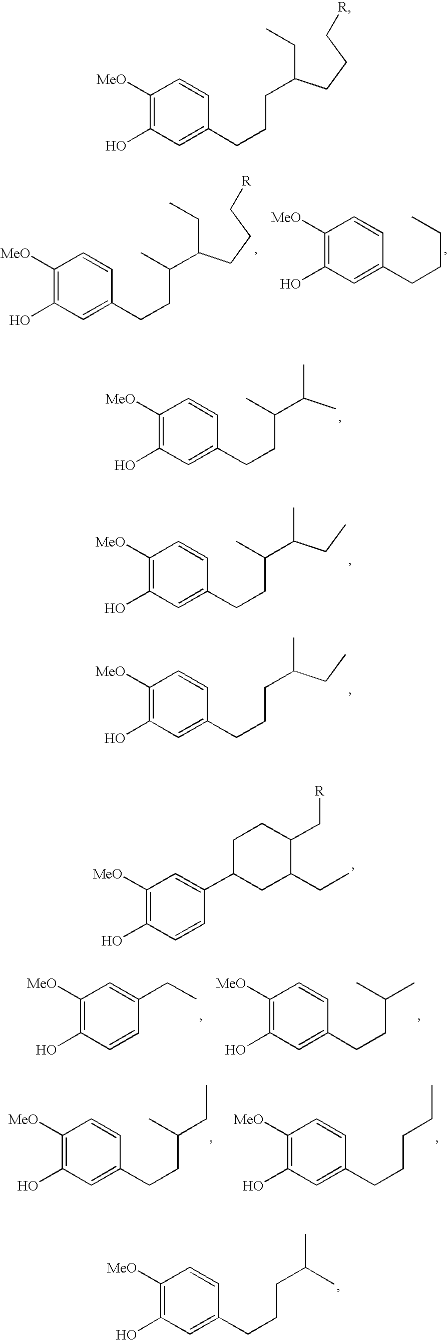 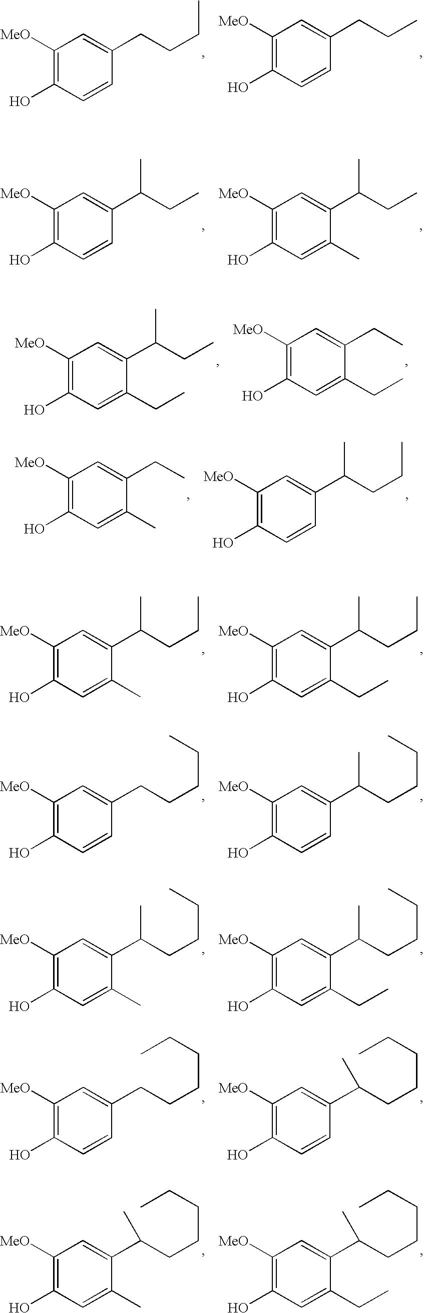 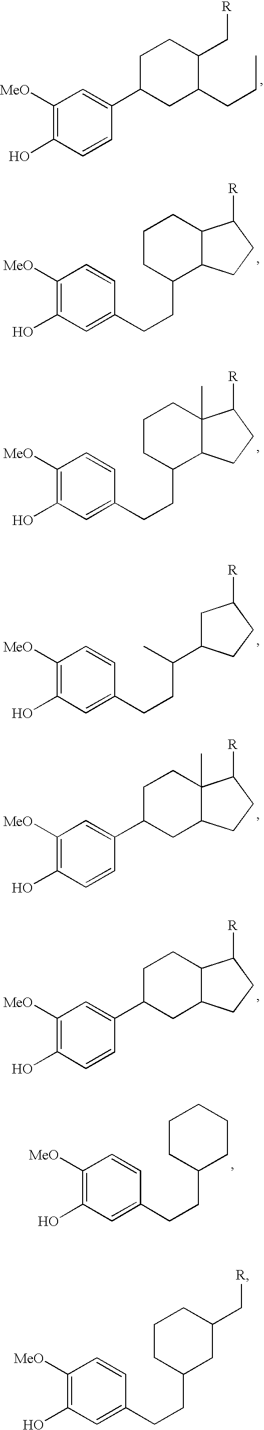 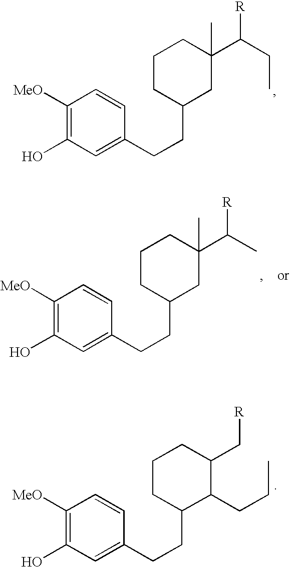
wherein R is selected from H, OH, ═CH₂ or ═CHCH₃.

10. The compound of claim 1, wherein the compound is selected from 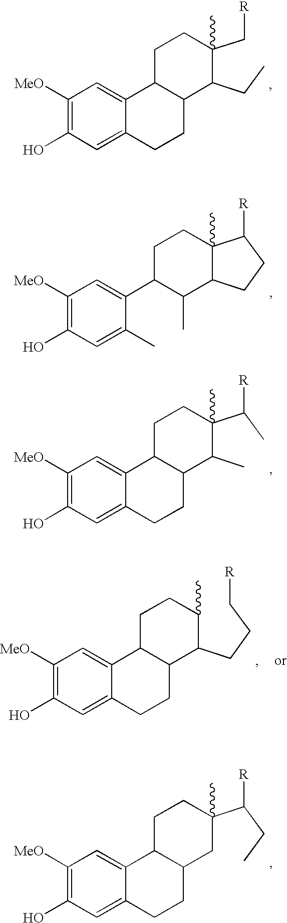
wherein R is selected from H, OH, ═CH₂ or ═CHCH₃.

11. The compound of claim 1, wherein the compound is:

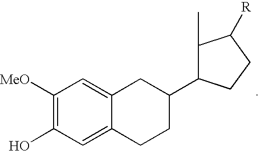

12. The compound of claim 1, wherein the compound is:

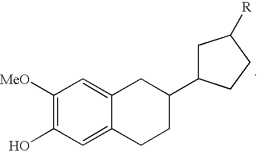

13. The compound of claim 1, wherein the compound is:

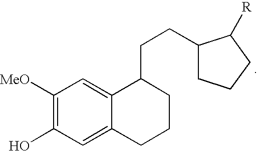

14. The compound of claim 1, wherein the compound is:

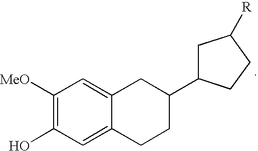

15. The compound of claim 1, wherein the compound is:

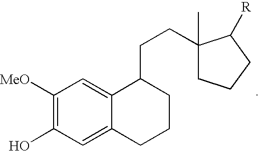

16. The compound of claim 1, wherein the compound is:

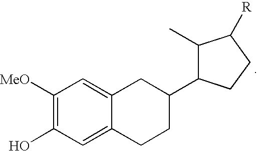

17. The compound of claim 1, wherein the compound is:

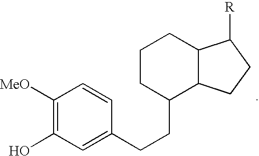

18. The compound of claim 1, wherein the compound is:

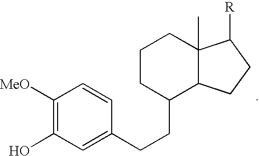

19. The compound of claim 1, wherein the compound is:

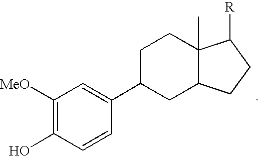

20. The compound of claim 1, wherein the compound is:

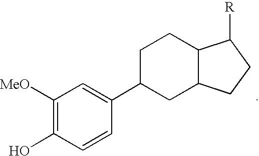

21. The compound of claim 1, wherein the compound is:

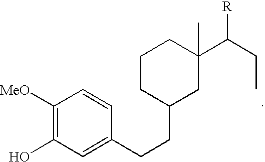

22. The compound of claim 1, wherein the compound is:

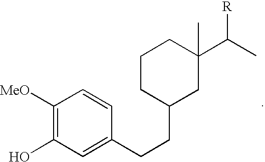

23. The compound of claim 1, wherein the compound is:

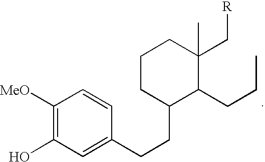

24. The compound of claim 1, wherein the compound is:

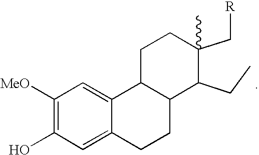

25. The compound of claim 1, wherein the compound is:

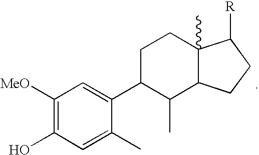

26. The compound of claim 1, wherein the compound is:

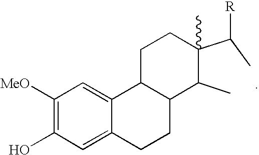

27. The compound of claim 1, wherein the compound is:

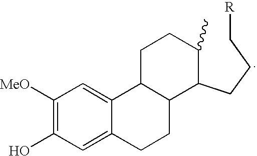

28. The compound of claim 1, wherein the compound is:

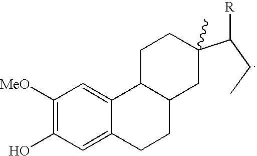

29. The compound of claim 1, wherein the compound is:

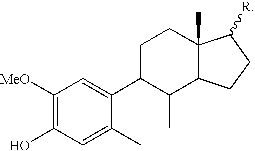

30. A pharmaceutical composition comprising:
a pharmaceutical carrier or excipient; and
a compound of the formula: 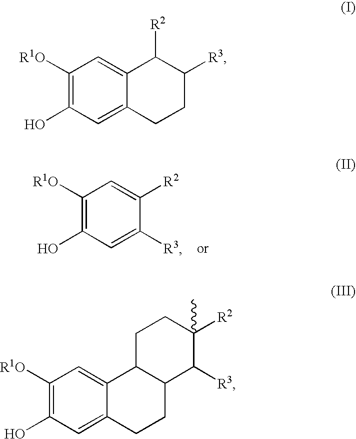
wherein R¹ is independently selected from an alkyl, aryl, substituted alkyl or substituted aryl with up to 13 carbon atoms; and
wherein R² and R³ are independently selected from
hydrogen;
halogen;
substituted or unsubstituted alkyl alkenyl, alkynyl, aromatic group, heterocyclic group, aryl, aralkyl, ether, amine, acyl, formyl, alkoxide, aryloxide, phosphate, trifluoroalkyl, thiol, alkyl thiol, aryl thiol, carboxylic acid, sulfonic acid, amino, alkyl amino, dialkyl amino, ester, cyano, sulfate, sulfonate, sulfone, sulfamate, imine, amide, alkyl amide, or dialkyl amide, any of which having up to 13 carbon atoms;
[NH₃]⁺X⁻, where X is selected from F, Cl, Br, or I;
a noncyclic heteroatom-containing group with up to 13 carbon atoms, wherein the heteroatom is selected from Si, N, P, O, or S;
wherein any substituted group comprises substituents selected from OH, F, Cl, Br, I, NH₂, OH, SH, OR, SiHₙR₃₋ₙ where n is an integer from 1-3 inclusive, NHR, NR₂, SR, or PR₂, where R is independently selected from an alkyl or aryl with up to 10 carbon atoms;
or a metabolite or a salt thereof;
in an amount effective upon administration in a daily dose, a daily sub-dose, or an appropriate fraction thereof to a human or an animal to inhibit undesired angiogenesis.

31. The pharmaceutical composition of claim 30, wherein the compound is

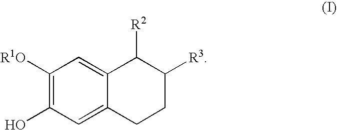

32. The pharmaceutical composition of claim 30, wherein the compound is

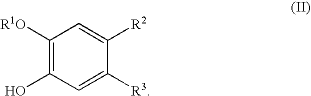

33. The pharmaceutical composition of claim 30, wherein the compound is

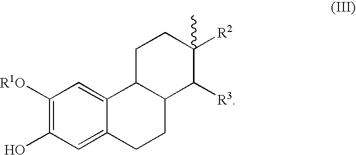

34. The pharmaceutical composition of claim 30, wherein only one of R² or R³ in structures (I), (II), and (III) is hydrogen.

35. The pharmaceutical composition of claim 30, wherein none of R² or R³ in structures (I), (II), and (III hydrogen.

36. The pharmaceutical composition of claim 30, wherein
R¹ is independently selected from an alkyl, aryl, substituted alkyl or substituted aryl with up to 13 carbon atoms; and
wherein R² and R³ are independently selected from
hydrogen;
substituted or unsubstituted alkyl, alkenyl, alkynyl, aromatic group, heterocyclic group, aryl, aralkyl, any of which having up to 13 carbon atoms; or
a noncyclic heteroatom-containing group with up to 13 carbon atoms, wherein the heteroatom is selected from Si, N, P, O, or S;
or a metabolite or a salt thereof.

37. The pharmaceutical composition of claim 30, wherein the compound is selected from 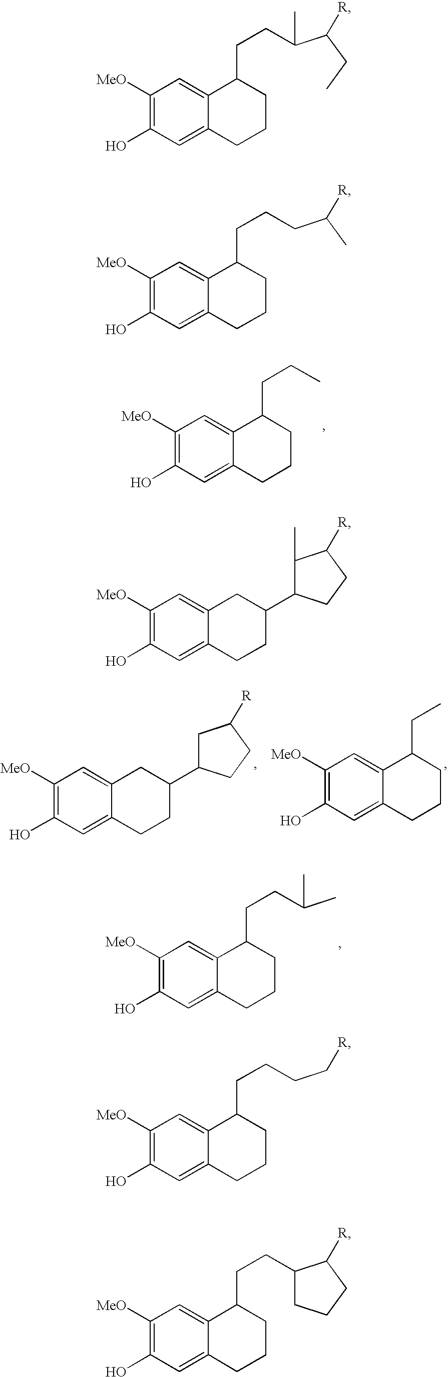 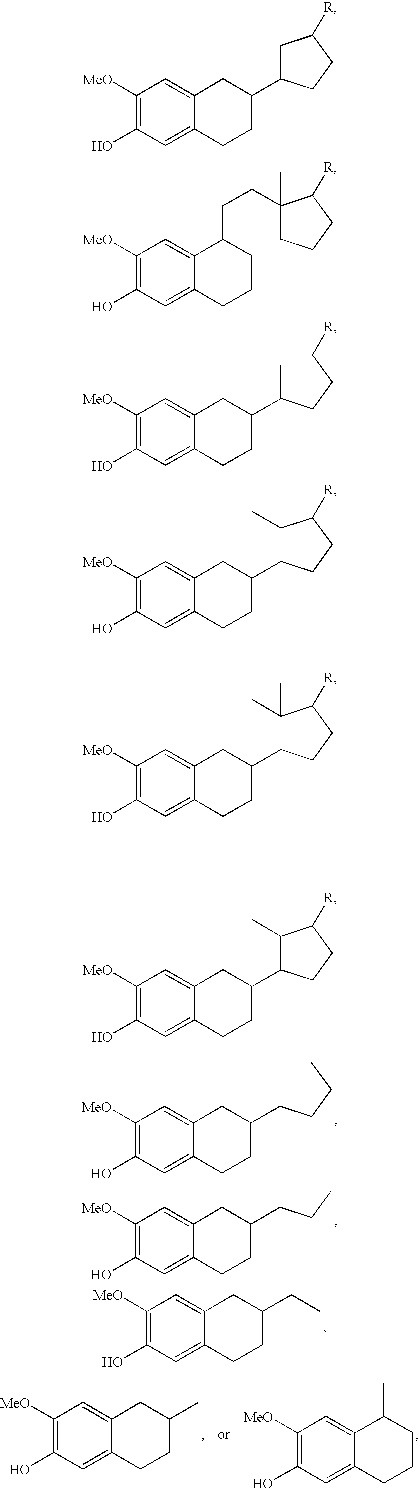
wherein R is selected from H, OH, ═CH₂ or ═CHCH₃.

38. The pharmaceutical composition of claim 30, wherein the compound is selected from 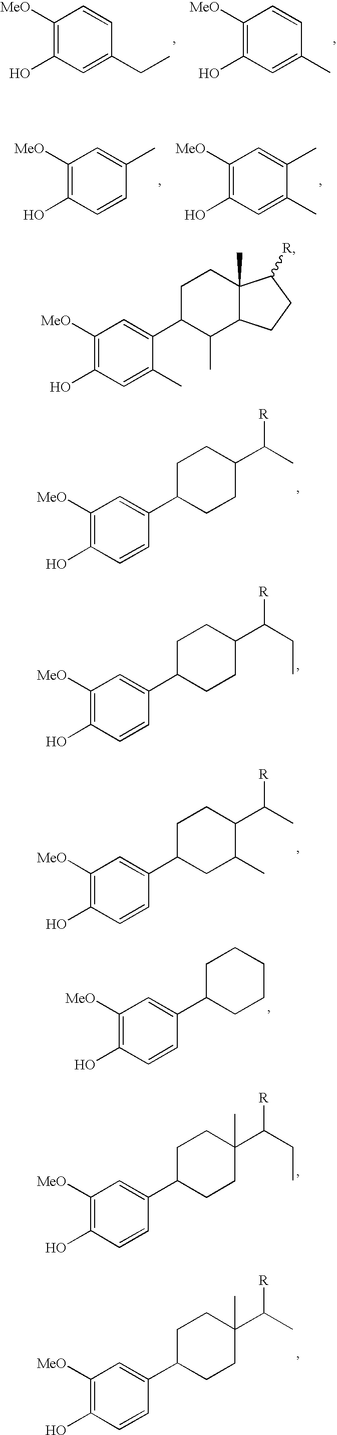 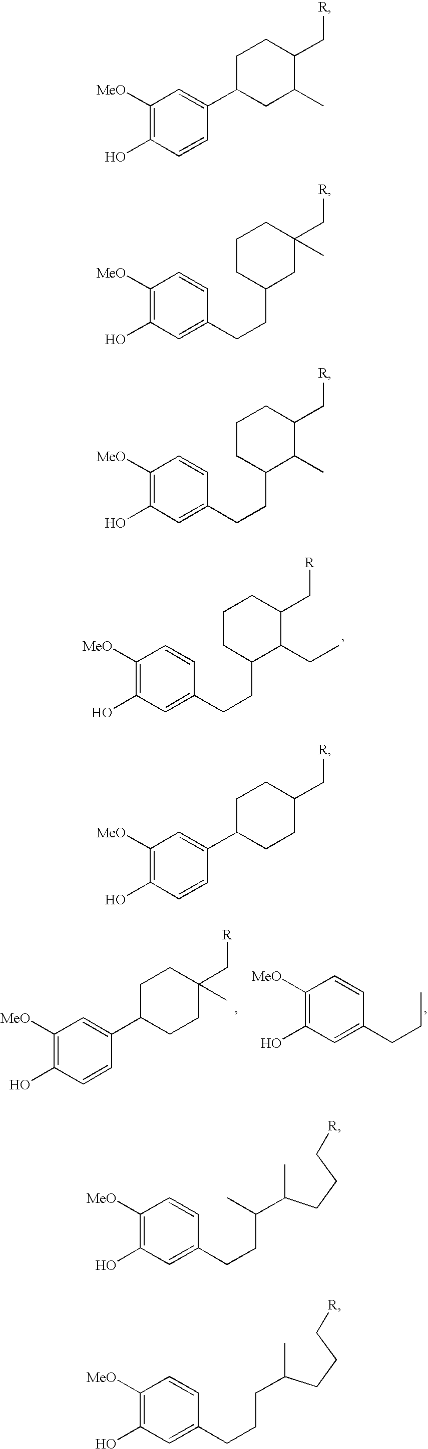 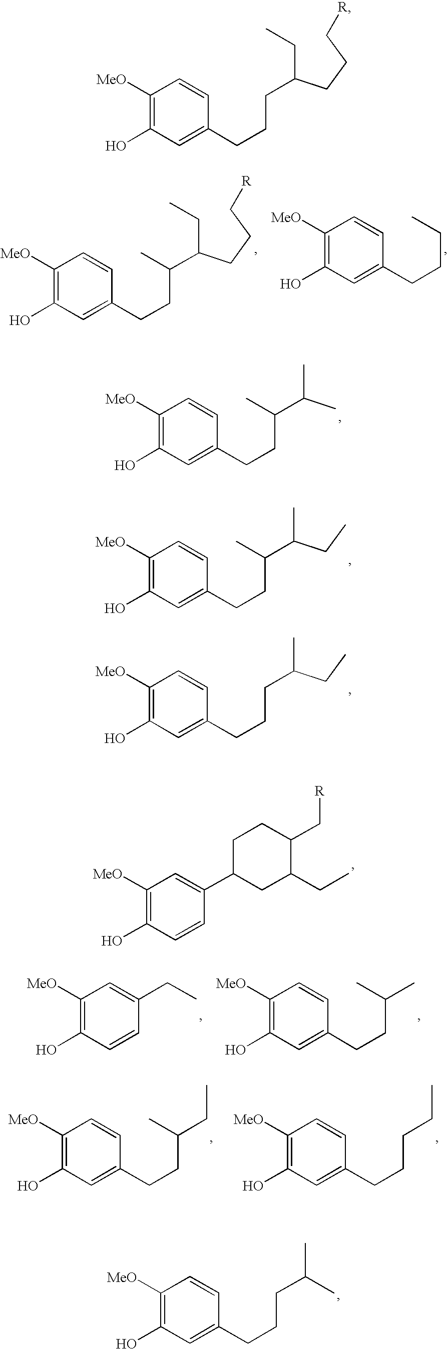 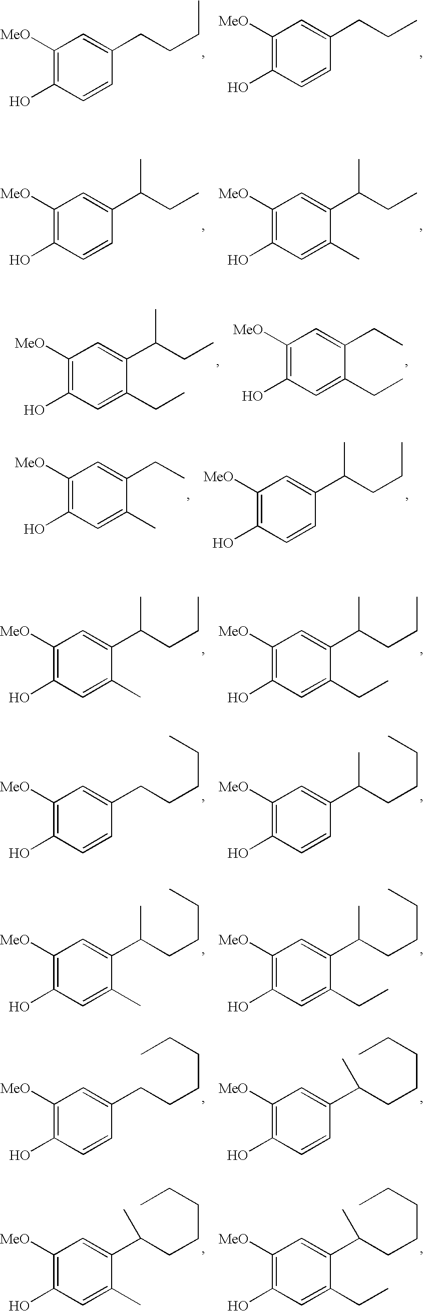 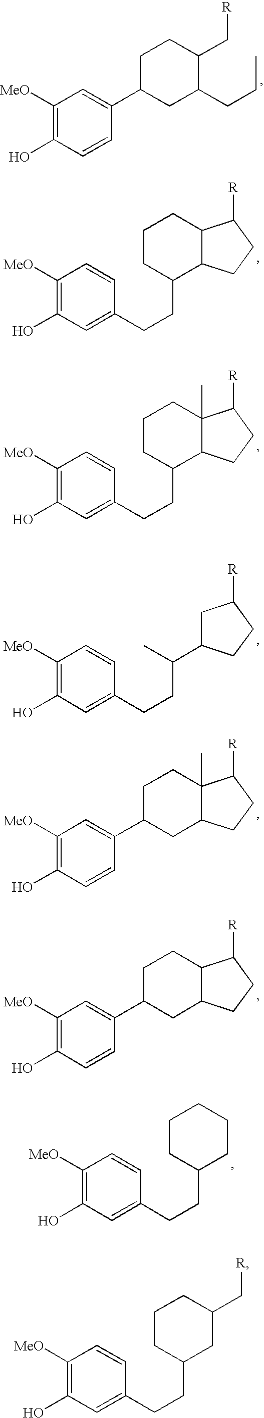 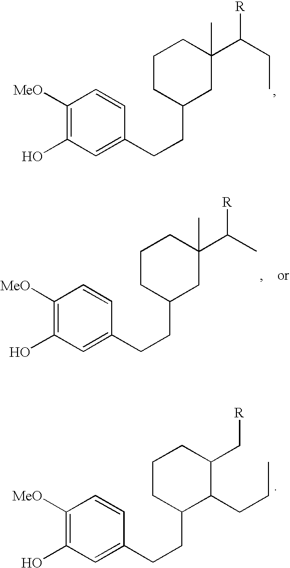
wherein R is selected from H, OH, ═CH₂ or ═CHCH₃.

39. The pharmaceutical composition of claim 30, wherein the compound is selected from 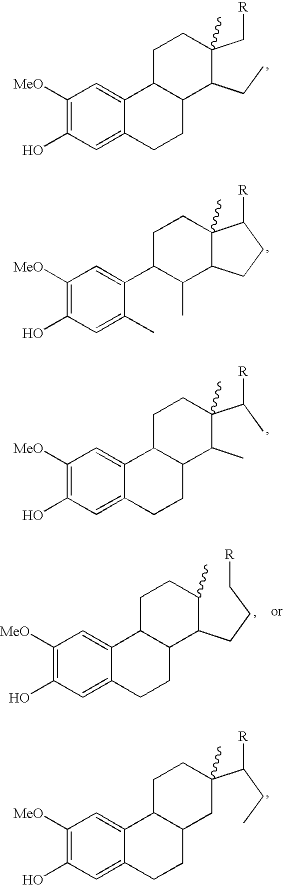
wherein R is selected from H, OH, ═CH₂ or ═CHCH₃.

40. The pharmaceutical composition of claim 30, wherein the compound is

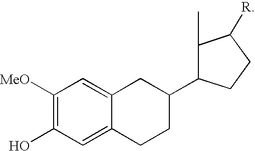

41. The pharmaceutical composition of claim 30, wherein the compound is

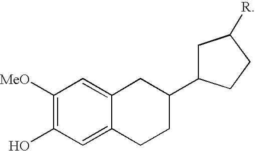

42. The pharmaceutical composition of claim 30, wherein the compound is

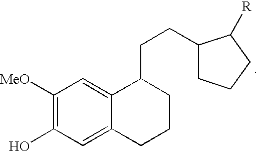

43. The pharmaceutical composition of claim 30, wherein the compound is

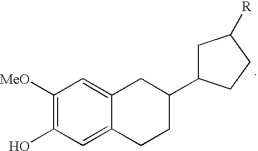

44. The pharmaceutical composition of claim 30, wherein the compound is

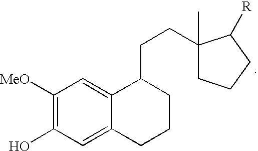

45. The pharmaceutical composition of claim 30, wherein the compound is

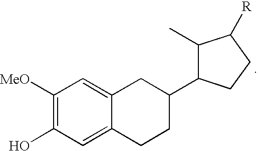

46. The pharmaceutical composition of claim 30, wherein the compound is

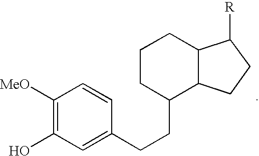

47. The pharmaceutical composition of claim 30, wherein the compound is

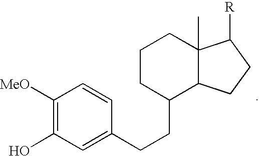

48. The pharmaceutical composition of claim 30, wherein the compound is

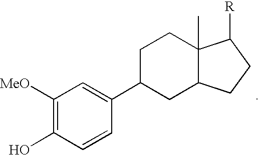

49. The pharmaceutical composition of claim 30, wherein the compound is

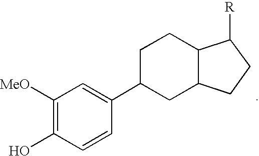

50. The pharmaceutical composition of claim 30, wherein the compound is

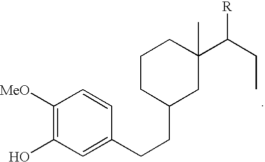

51. The pharmaceutical composition of claim 30, wherein the compound is

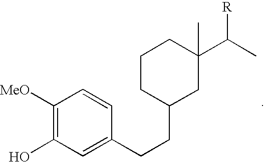

52. The pharmaceutical composition of claim 30, wherein the compound is

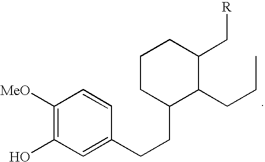

53. The pharmaceutical composition of claim 30, wherein the compound is:

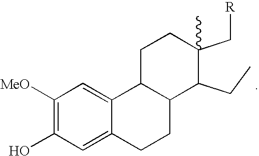

54. The pharmaceutical composition of claim 30, wherein the compound is:

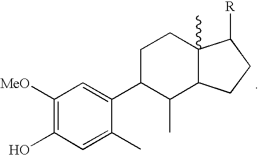

55. The pharmaceutical composition of claim 30, wherein the compound is:

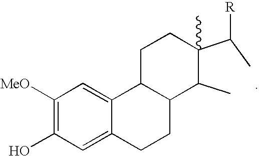

56. The pharmaceutical composition of claim 30, wherein the compound is:

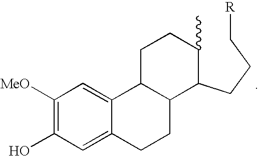

57. The pharmaceutical composition of claim 30, wherein the compound is:

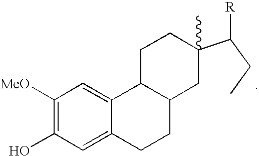

58. The pharmaceutical composition of claim 30, wherein the compound is:

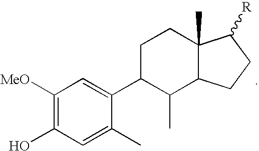

59. The pharmaceutical composition of claim 30, wherein the daily dose is between approximately 0.01 and 300 mg/kg/day.

60. The pharmaceutical composition of claim 30, wherein the daily dose is between approximately 0.05 and 50 mg/kg/day.

61. The pharmaceutical composition of claim 30, wherein the daily dose is between approximately 0.1 and 10 mg/kg/day.

62. The pharmaceutical composition of claim 30, wherein the daily dose is between approximately 0.1 and 1 mg/kg/day.

63. The pharmaceutical composition of claim 30, wherein the composition is in the form of a tablet, capsule, a lozenge, a cachet, a solution, a suspension, an emulsion, a powder, a granule, an aerosol, a suppository, a spray, a pastille, an ointment, a cream, a paste, a foam, a gel, a tampon, a bolus, a mouthwash, a transdermal patch, or a pessary.

64. The pharmaceutical composition of claim 30, further comprising an additive selected from an anti-oxidant, a buffer, a bacteriostat, a solute, a suspending agent, a thickening agent, a flavoring agent, a gelatin, glycerine, a diluent, a binder, a lubricant, a preservative, a surface active agent, a dispersing agent, a biodegradable polymer, or any combination thereof.

65. The pharmaceutical composition of claim 30, wherein the undesired angiogenesis is associated with diabetic retinopathy, retinopathy of prematurity, corneal graft rejection; neovascular glaucoma, retrolental fibroplasias; epidemic keratoconjunctivitis; Vitamin A deficiency; contact lens overwear; atopic keratitis; superior limbic keratitis; pterygium keratitis sicca; sjogren's syndrome; acne rosacea; phylectenulosis; syphilis; Mycobacteria infections; lipid degeneration; chemical burns; bacterial ulcers; fungal ulcers; Herpes simplex infections; Herpes zoster infections; protozoan infections; Kaposi's sarcoma; Mooren's ulcer; Terrien's marginal degeneration; marginal keratolysis; trauma; rheumatoid arthritis; systemic lupus; polyarteritis; Wegener's syndrome; sarcoidosis; Scleritis; Stevens-Johnson disease; radial keratotomy; macular degeneration; sickle cell anemia; sarcoid; pseudoxanthoma elasticum; Paget's disease; vein occlusion; artery occlusion; carotid obstructive disease; chronic uveitis; chronic vitritis; Lyme's disease; Eales' disease; Beheet's disease; myopia; optic pits; Stargardt's disease; pars planitis; chronic retinal detachment; hyperviscosity syndromes; toxoplasmosis; post-laser complications; abnormal proliferation of fibrovascular or fibrous tissue; hemangiomas; Osler-Weber-Rendu disease; solid tumors; blood-borne tumors; acquired immune deficiency syndrome; ocular neovascular disease; age-related macular degeneration; osteoarthritis; diseases caused by chronic inflammation; Crohn's disease; ulcerative colitis; tumors of rhabdomyosarcoma; tumors of retinoblastoma; Ewing's sarcoma; with neuroblastoma; tumors of osteosarcoma; leukemia; psoriasis; atherosclerosis; pemphigoid; infections causing retinitis or choroiditis; presumed ocular histoplasmosis; Best's disease; proliferative vitreoretinopathy; Bartonellosis; acoustic neuroma; neurofibroma; trachoma; or pyogenic granulomas.

66. A method of treating a condition selected from an ocular condition, an inflammatory or immune mediated disease, an infectious disease, a cancerous disease, a blood or blood vessel disease, a skin condition, or a tumor in a human or an animal comprising administering to the human or animal a composition comprising a compound having the formula: 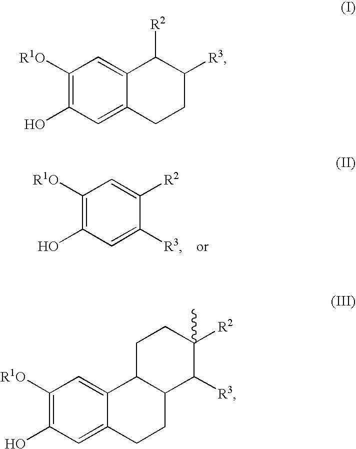
wherein R¹ is independently selected from an alkyl, aryl, substituted alkyl or substituted aryl with up to 13 carbon atoms; and
wherein R² and R³ are independently selected from
hydrogen;
halogen;
substituted or unsubstituted alkyl, alkenyl, alkynyl, aromatic group, heterocyclic group, aryl, aralkyl, ether, amine, acyl, formyl, alkoxide, aryloxide, phosphate, trifluoroalkyl, thiol, alkyl thiol, aryl thiol, carboxylic acid, sulfonic acid, amino, alkyl amino, dialkyl amino, ester, cyano, sulfate, sulfonate, sulfone, sulfamate, imine, amide, alkyl amide, or dialkyl amide, any of which having up to 13 carbon atoms;
[NH₃]⁺X⁻, where X is selected from F, Cl, Br, or I;
a noncyclic heteroatom-containing group with up to 13 carbon atoms, wherein the heteroatom is selected from Si, N, P, O, or S;
wherein any substituted group comprises substituents selected from OH, F, Cl, Br, I, NH₂, OH, SH, OR, SiHₙR₃₋ₙ, where n is an integer from 1-3 inclusive, NHR, NR₂, SR, or PR₂, where R is independently selected from an alkyl or aryl with up to 10 carbon atoms;
or a metabolite or a salt thereof;
in an amount effective to treat the condition.

67. The method of claim 66, wherein the compound is

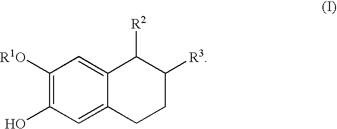

68. The method of claim 66, wherein the compound is

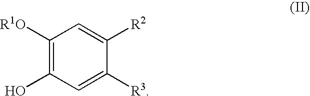

69. The method of claim 66, wherein the compound is

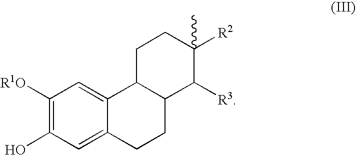

70. The method of claim 66, wherein only one of R² or R³ in structures (I), (II), and (III) is hydrogen.

71. The method of claim 66, wherein none of R² or R³ in structures (I), (II), and (III) is hydrogen.

72. The method of claim 66, wherein
R¹ is independently selected from an alkyl, aryl, substituted alkyl or substituted aryl with up to 13 carbon atoms; and
wherein R² and R³ are independently selected from
hydrogen;
substituted or unsubstituted alkyl, alkenyl, alkynyl, aromatic group, heterocyclic group, aryl, aralkyl, any of which having up to 13 carbon atoms; or
a noncyclic heteroatom-containing group with up to 13 carbon atoms, wherein the heteroatom is selected from Si, N, P, O, or S;
or a metabolite or a salt thereof.

73. The method of claim 66, wherein the compound is selected from

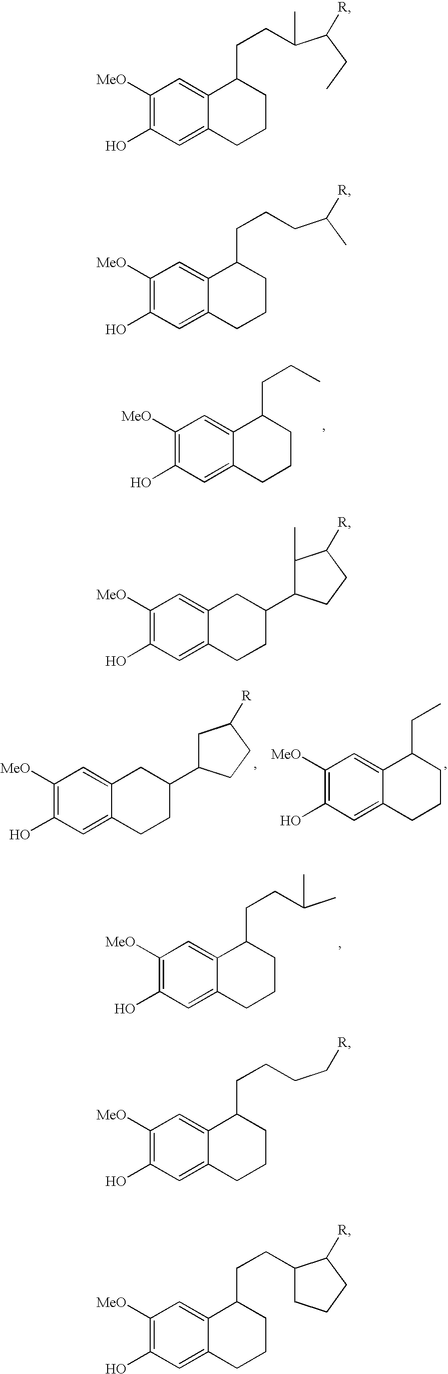

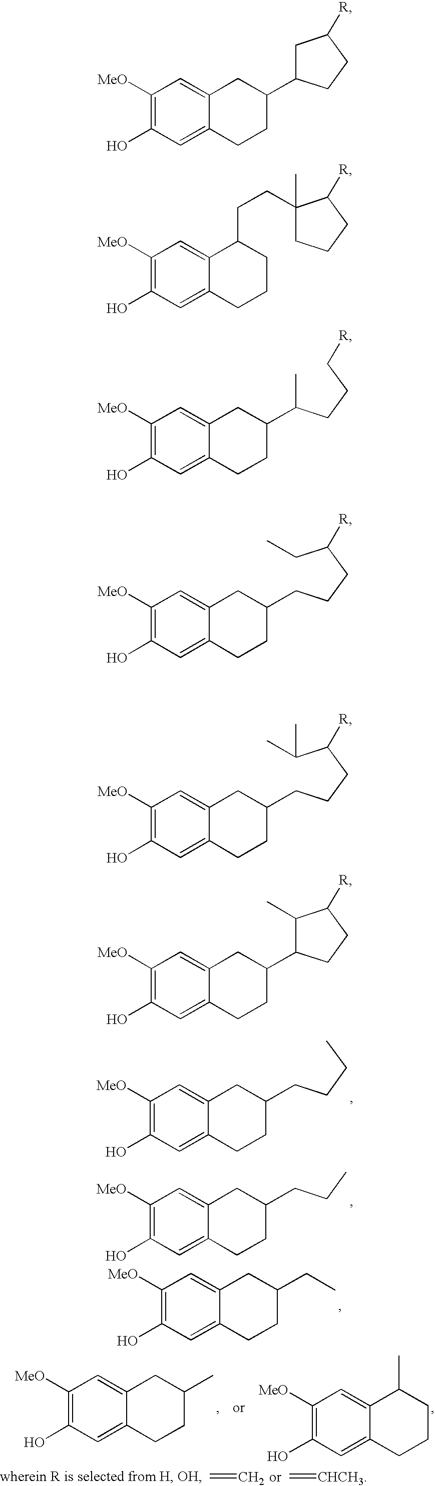

74. The method of claim 66, wherein the compound is selected from 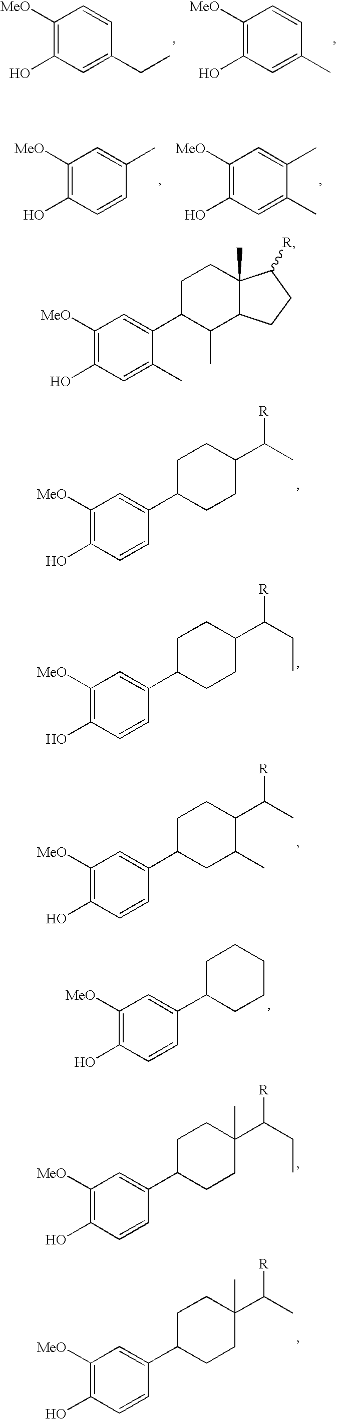 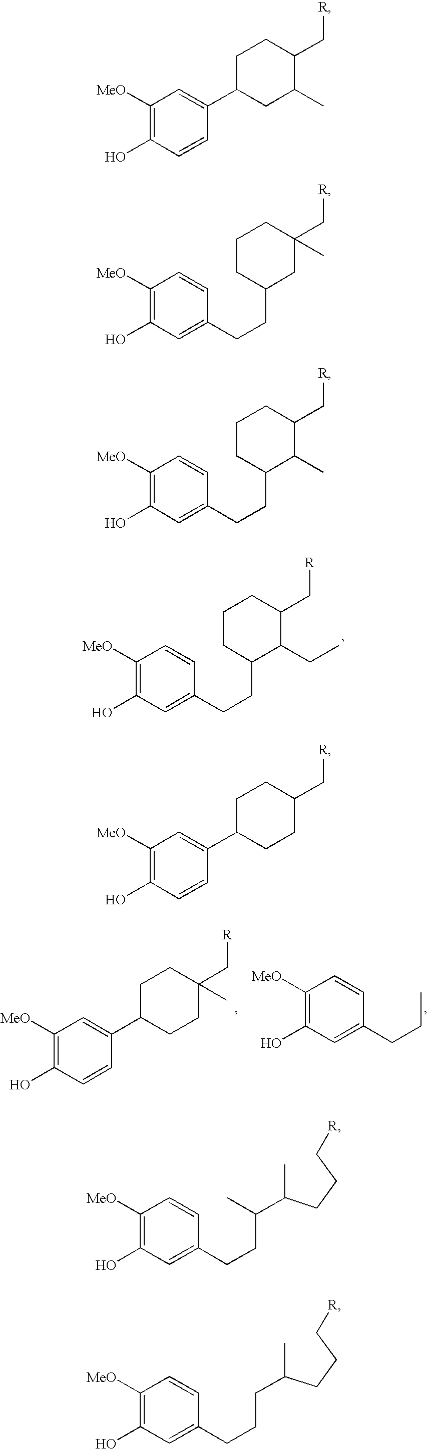 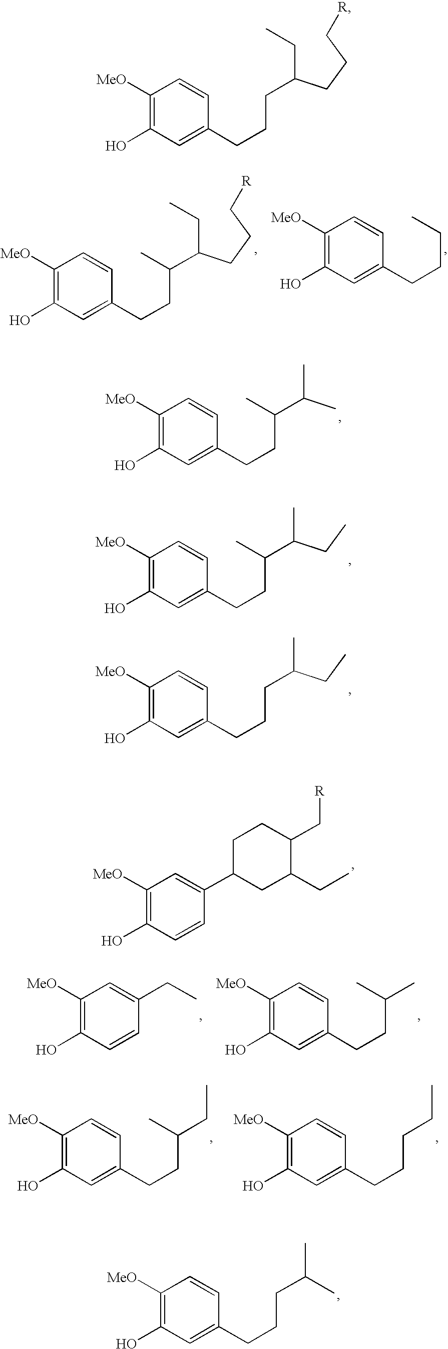 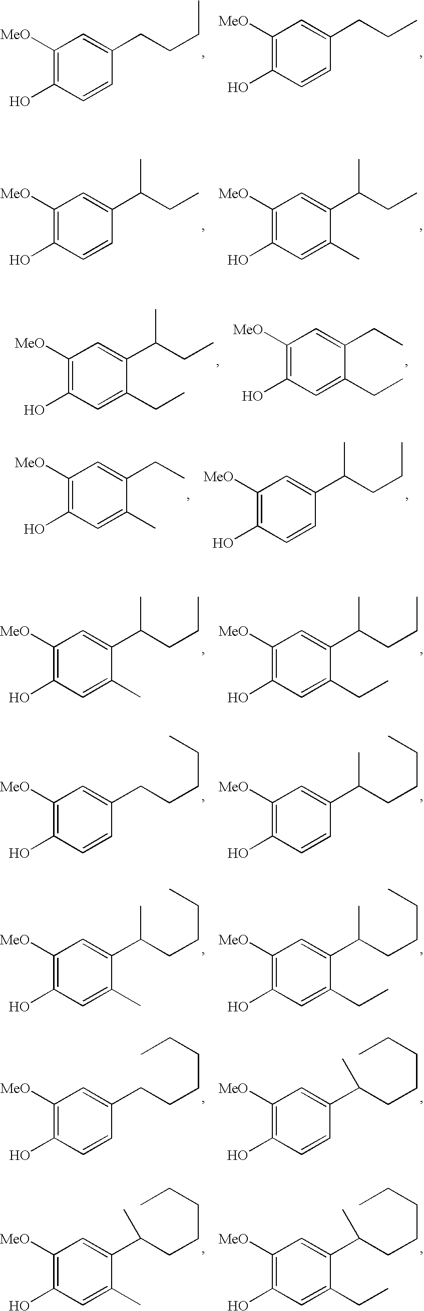 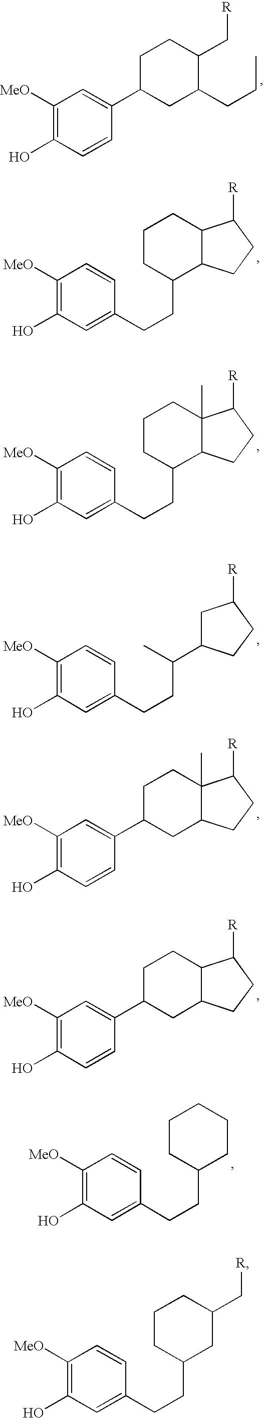 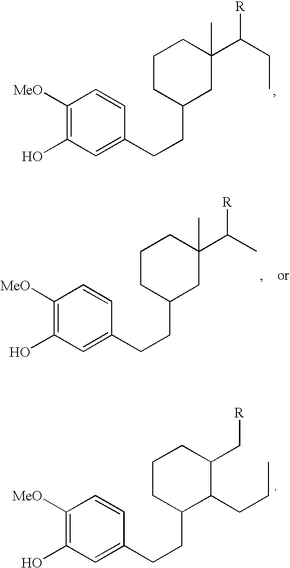
wherein R is selected from H, OH, ═CH₂ or ═CHCH₃.

75. The method of claim 66, wherein the compound is selected from 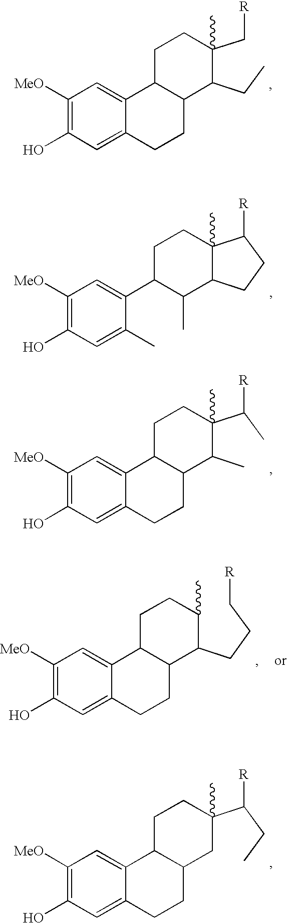
wherein R is selected from H, OH, ═CH₂ or ═CHCH₃.

76. The method of claim 66, wherein the compound is

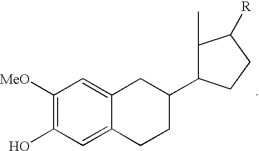

77. The method of claim 66, wherein the compound is

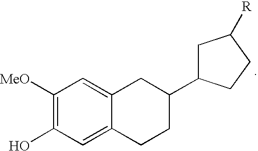

78. The method of claim 66, wherein the compound is

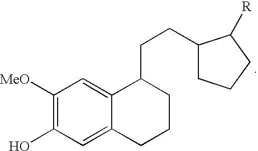

79. The method of claim 66, wherein the compound is

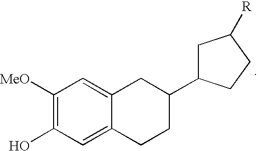

80. The method of claim 66, wherein the compound is

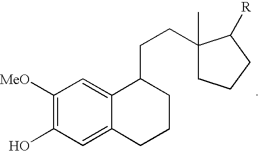

81. The method of claim 66, wherein the compound is

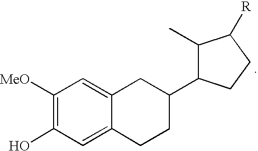

82. The method of claim 66, wherein the compound is

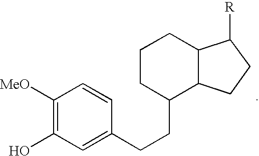

83. The method of claim 66, wherein the compound is

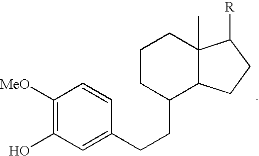

84. The method of claim 66, wherein the compound is

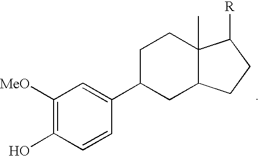

85. The method of claim 66, wherein the compound is

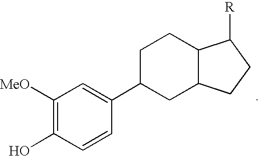

86. The method of claim 66, wherein the compound is

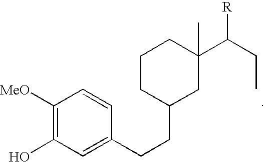

87. The method of claim 66, wherein the compound is

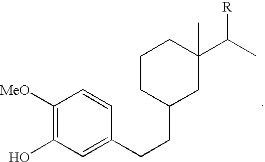

88. The method of claim 66, wherein the compound is

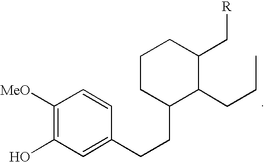

89. The method of claim 66, wherein the compound is

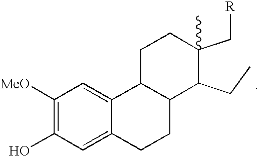

90. The method of claim 66, wherein the compound is

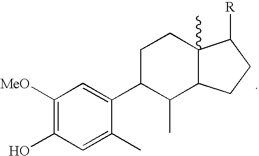

91. The method of claim 66, wherein the compound is

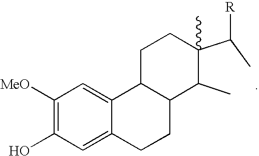

92. The method of claim 66, wherein the compound is

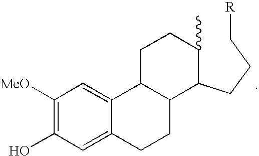

93. The method of claim 66, wherein the compound is

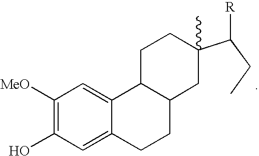

94. The method of claim 66, wherein the compound is

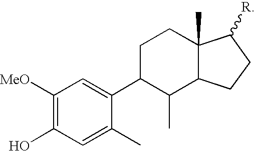

95. The method of claim 66, wherein the daily dose is between approximately 0.01 and 300 mg/kg/day.

96. The method of claim 66, wherein the daily dose is between approximately 0.05 and 50 mg/kg/day.

97. The method of claim 66, wherein the daily dose is between approximately 0.1 and 10 mg/kg/day.

98. The method of claim 66, wherein the daily dose is between approximately 0.1 and 1 mg/kg/day.

99. The method of claim 66, wherein the composition is in the form of a tablet, capsule, a lozenge, a cachet, a solution, a suspension, an emulsion, a powder, a granule, an aerosol, a suppository, a spray, a pastille, an ointment, a cream, a paste, a foam, a gel, a tampon, a bolus, a mouthwash, a transdermal patch, or a pessary.

100. The method of claim 66, further comprising an additive selected from an anti-oxidant, a buffer, a bacteriostat, a solute, a suspending agent, a thickening agent, a flavoring agent, a gelatin, glycerine, a diluent, a binder, a lubricant, a preservative, a surface active agent, a dispersing agent, a biodegradable polymer, or any combination thereof.

101. The method of claim 66, wherein the undesired angiogenesis is associated with diabetic retinopathy, retinopathy of prematurity, corneal graft rejection; neovascular glaucoma, retrolental fibroplasias; epidemic keratoconjunctivitis; Vitamin A deficiency; contact lens overwear; atopic keratitis; superior limbic keratitis; pterygium keratitis sicca; sjogren's syndrome; acne rosacea; phylectenulosis; syphilis; Mycobacteria infections; lipid degeneration; chemical bums; bacterial ulcers; fungal ulcers; Herpes simplex infections; Herpes zoster infections; protozoan infections; Kaposi's sarcoma; Mooren's ulcer; Terrien's marginal degeneration; marginal keratolysis; trauma; rheumatoid arthritis; systemic lupus; polyarteritis; Wegener's syndrome; sarcoidosis; Scleritis; Stevens-Johnson disease; radial keratotomy; macular degeneration; sickle cell anemia; sarcoid; pseudoxanthoma elasticum; Paget's disease; vein occlusion; artery occlusion; carotid obstructive disease; chronic uveitis; chronic vitritis; Lyme's disease; Eales' disease; Behcet's disease; myopia; optic pits; Stargardt's disease; pars planitis; chronic retinal detachment; hyperviscosity syndromes; toxoplasmosis; post-laser complications; abnormal proliferation of fibrovascular or fibrous tissue; hemangiomas; Osler-Weber-Rendu disease; solid tumors; blood-borne tumors; acquired immune deficiency syndrome; ocular neovascular disease; age-related macular degeneration; osteoarthritis; diseases caused by chronic inflammation; Crohn's disease; ulcerative colitis; tumors of rhabdomyosarcoma; tumors of retinoblastoma; Ewing's sarcoma; with neuroblastoma; tumors of osteosarcoma; leukemia; psoriasis; atherosclerosis; pemphigoid; infections causing retinitis or choroiditis; presumed ocular histoplasmosis; Best's disease; proliferative vitreoretinopathy; Bartonellosis; acoustic neuroma; neurofibroma; trachoma; or pyogenic granulomas.

102. The method of claim 66, wherein the composition is in the form of a tablet, capsule, a lozenge, a cachet, a solution, a suspension, an emulsion, a powder, a granule, an aerosol, a suppository, a spray, a pastille, an ointment, a cream, a paste, a foam, a gel, a tampon, a bolus, a mouthwash, a transdermal patch, or a pessary.

103. The method of claim 66, further comprising an additive selected from an anti-oxidant, a buffer, a bacteriostat, a solute, a suspending agent, a thickening agent, a flavoring agent, a gelatin, glycerine, a diluent, a binder, a lubricant, a preservative, a surface active agent, a dispersing agent, a biodegradable polymer, or any combination thereof.

104. The method of claim 66, wherein the undesired angiogenesis is associated with diabetic retinopathy, retinopathy of prematurity, corneal graft rejection; neovascular glaucoma, retrolental fibroplasias; epidemic keratoconjunctivitis; Vitamin A deficiency; contact lens overwear; atopic keratitis; superior limbic keratitis; pterygium keratitis sicca; sjogren's syndrome; acne rosacea; phylectenulosis; syphilis; Mycobacteria infections; lipid degeneration; chemical bums; bacterial ulcers; fungal ulcers; Herpes simplex infections; Herpes zoster infections; protozoan infections; Kaposi's sarcoma; Mooren's ulcer; Terrien's marginal degeneration; marginal keratolysis; trauma; rheumatoid arthritis; systemic lupus; polyarteritis; Wegener's syndrome; sarcoidosis; Scleritis; Stevens-Johnson disease; radial keratotomy; macular degeneration; sickle cell anemia; sarcoid; pseudoxanthoma elasticum; Paget's disease; vein occlusion; artery occlusion; carotid obstructive disease; chronic uveitis; chronic vitritis; Lyme's disease; Eales' disease; Behcet's disease; myopia; optic pits; Stargardt's disease; pars planitis; chronic retinal detachment; hyperviscosity syndromes; toxoplasmosis; post-laser complications; abnormal proliferation of fibrovascular or fibrous tissue; hemangiomas; Osler-Weber-Rendu disease; solid tumors; blood-borne tumors; acquired immune deficiency syndrome; ocular neovascular disease; age-related macular degeneration; osteoarthritis; diseases caused by chronic inflammation; Crohn's disease; ulcerative colitis; tumors of rhabdomyosarcoma; tumors of retinoblastoma; Ewing's sarcoma; with neuroblastoma; tumors of osteosarcoma; leukemia; psoriasis; atherosclerosis; pemphigoid; infections causing retinitis or choroiditis; presumed ocular histoplasmosis; Best's disease; proliferative vitreoretinopathy; Bartonellosis; acoustic neuroma; neurofibroma; trachoma; or pyogenic granulomas.
